(19) United States
(12) Patent Application Publication
(10) Pub. No.: US 2011/0146702 A1
(43) Pub. Date: Jun. 23, 2011

(54) EXTENDING COSMETIC COMPOSITION COMPRISING BEHENYL ALCOHOL AS THICKENER

(75) Inventor: Olivier RAINEAU, Paris (FR)

(73) Assignee: L'OREAL, Paris (FR)

(21) Appl. No.: 12/966,387

(22) Filed: Dec. 13, 2010

Related U.S. Application Data
(60) Provisional application No. 61/306,974, filed on Feb. 23, 2010.
(60) Provisional application No. 61/306,973, filed on Feb. 23, 2010.

(30) Foreign Application Priority Data
Dec. 17, 2009 (FR) 09 59140
Dec. 17, 2009 (FR) 09 59147

(51) Int. Cl. A45D 33/00; A61K 8/92; A61Q 1/10

(52) U.S. Cl. 132/293; 424/70.7

(57) ABSTRACT

The present invention relates to a cosmetic composition for make-up and/or care of keratin fibres comprising at least one emulsifying system free from triethanolamine stearate, characterized in that it contains behenyl alcohol, and in that the texture value measured by texture analysis counting from preparation of said composition, namely 24 hours after manufacture of the composition, is greater than 20 g at room temperature.

The present invention relates to the field of cosmetic compositions for make-up and/or care of keratin fibres, comprising an emulsifying system free from triethanolamine stearate. Advantageously, these compositions possess extending properties.

The texture of such compositions, and in particular the stability of the texture over time, is decisive for the user.

In fact, it has been observed in the field of mascaras, and notably so-called “extending” mascaras, and more particularly mascaras comprising cetyl alcohol as thickener, that the variation in the texture of the latter is detrimental to the quality of make-up as they are used.

In other words, during storage and after the packaging has been opened, the composition “ages” and its texture tends to increase to the detriment of the application qualities that are normally required, namely easy deposition of the product on the eyelashes.

This change in texture can give mascaras that adhere less and are deposited less.

Now, it is also known that the material deposited on the keratin fibres or load, depends not only on its thick texture, but also on the stiffness of the applicator, which can notably be a brush. More precisely, the more flexible the applicator, the more material is deposited. However, because of the increase in texture of the compositions over time, it often becomes necessary to use applicators comprising stiffer application elements such as bristles or teeth in order to counteract this increase in texture. In fact, when the applicator/texture combination of the composition is not suitable, we encounter the defect known as “Christmas-tree brush”, where the bristles of the applicator, in this case the brush, become flattened during passage through the wiper and do not stand up again as they have insufficient rigidity.

Consequently, prior to marketing, a study is generally conducted regarding the match between the texture of the composition and the stiffness of the applicator counting from preparation of the composition once the latter has aged. Owing to this constraint, applicators of greater stiffness than would theoretically be required for obtaining the desired loading are then chosen, to the detriment of applicators that are more flexible, even though the latter are more suitable with respect to application.

Thus, there is a need for cosmetic compositions, and notably extending mascaras, offering a relatively high texture after manufacture, which should not be minimized in anticipation of the change in texture over time, mentioned above, permitting the use of applicators that are more flexible than those commonly employed for equivalent compositions not according to the present invention.

The inventors have demonstrated the advantages connected with the use of behenyl alcohol, notably as thickener, and notably replacing, completely or partially, the cetyl alcohol commonly used for these same purposes, to overcome the aforementioned drawbacks.

The present invention thus relates to a cosmetic composition for make-up and/or care of keratin fibres comprising at least one emulsifying system free from triethanolamine stearate, characterized in that it contains behenyl alcohol, and in that the texture value measured by texture analysis counting from preparation of said composition, namely 24 hours after manufacture of the composition, is greater than 20 g at room temperature.

More particularly, the present invention relates to composition for make-up and/or care of keratin fibres comprising at least one emulsifying system free from triethanolamine stearate, characterized in that it contains at least one pigment and behenyl alcohol, and in that the texture value measured by texture analysis, according to the method of measurement of texture described in the present application, counting from preparation of said composition, namely 24 hours after manufacture of the composition, is greater than 20 g at room temperature,

said behenyl alcohol being present at a content greater than or equal to 1 wt. % relative to the total weight of the composition,
said emulsifying system comprising at least one surfactant selected from:
i) an alkali metal alkyl phosphate or phosphine oxide of formula (R—O)ₙ—P═O—(O⁻M)ₘ with R representing a linear or branched C₈-C₂₂ alkyl group, such as cetyl, n being equal to 1, 2 or 3 and in being equal to 0, 1 or 2, with m+n being equal to 3 and M representing a hydrogen atom or an alkali metal or alkaline-earth metal, preferably n=1 and m=2, and M is an alkali metal, such as sodium or potassium,
ii) a polyethoxylated alcohol of formula R′—(OCH₂CH₂)ₚ—OH with R′ representing a linear or branched C₁-C₃₀ alkyl and in particular represents CH₃—(CH₂)₁₇— and p representing an integer between 1 and 30 inclusive, preferably between 2 and 20; such as steareth-20 and steareth-2,
iii) a salt of glutamic acid of formula R—CONH—C(COO⁻M)-C₂H₄—COO-M′ with R representing a linear or branched C₈-C₂₂ alkyl group such as stearyl and M′ representing an alkali metal or alkaline-earth metal, and
iv) an alkyl glucoside obtained by condensation of glucose and of linear or branched C₈-C₂₂ fatty alcohols such as a cetyl and stearyl mixture called cetearyl.

The method of measurement of texture value is advantageously that described in the description given below.

The present invention also relates to the use of behenyl alcohol, notably as thickener, in a cosmetic composition for make-up and/or care of keratin fibres, and comprising at least one emulsifying system free from triethanolamine stearate, for improving the stability of the texture of said composition.

The present invention further relates to a kit for packaging and application comprising a container containing a composition as defined previously and an applicator configured for applying said composition on a keratinous material, and in particular on keratin fibres, such as the eyelashes or eyebrows, said applicator comprising application elements, such as bristles or teeth, having a hardness between 20 Shore A and 40 Shore D.

According to another aspect, the present invention also relates to a method of coating, and notably of make-up and/or care, of keratin fibres, such as the eyelashes or eyebrows, comprising a stage of application of a composition as defined previously on said keratin fibres.

As well as the need for extending mascaras, there is also a need for cosmetic compositions, and notably mascaras, offering a texture that is stable over time, in other words a controlled and limited variation in texture, ensuring reproducible application on the keratin fibres.

Thus, according to a second aspect, the present invention also relates to a cosmetic composition for make-up and/or care of keratin fibres comprising at least one emulsifying system free from triethanolamine stearate, characterized in that it contains behenyl alcohol, and in that the change in texture measured by texture analysis in a period of 60 days at 45° C. counting from preparation of said composition, namely 24 hours after manufacture of the composition, is less than 100%, the change in texture being defined by:

$$\frac{{T}_{60 j} - {T}_{0}}{{T}_{0}} \times 100$$

where T₆₀ⱼ is the measurement from texture analysis at 60 days, and

T₀ is the measurement from texture analysis taken 24 hours after manufacture of the preparation of the composition.

Still according to this second object, the present invention relates to the use of behenyl alcohol, notably as thickener, in a cosmetic composition for make-up and/or care of keratin fibres, and comprising at least one emulsifying system free from triethanolamine stearate, for improving the stability of the texture of said composition.

Still according to this second object, the present invention further relates to a kit for packaging and application comprising a container containing a composition as defined previously according to the second aspect, and an applicator configured for applying said composition on a keratinous material, and in particular on keratin fibres, such as the eyelashes or eyebrows, said applicator comprising application elements, such as bristles or teeth, having a hardness between 20 Shore A and 40 Shore D.

Still according to this second aspect, the present invention also relates to a method of coating, and notably of make-up and/or of care of keratin fibres, such as the eyelashes or eyebrows, comprising a stage of application of a composition as defined previously according to the second aspect on said keratin fibres.

For simplicity, the terminology ‘first aspect of the invention’ and ‘second aspect of the invention’ is used in the rest of the description for denoting the two particular embodiments described above.

If it is not specified, the description relates indiscriminately to both aspects of the invention.

Method of Texture Measurement

The texture of mascaras is measured according to the following protocol:

The measuring instrument is a TA-XT2 sold by the company Rheo, equipped with a force measurement cell of 5 kg and a cylindrical spindle with diameter of 12.7 mm (½ inch) made of Delrin. The mascara is thermostatically controlled at 20° C. Then it is placed in excess in a container with diameter of 60 mm and depth of 22 mm using a metal spatula. The product is spread to avoid any air pockets but without manipulating it so as not to destructure it. Then the container is levelled with a spatula so that the surface is as even as possible. The container is then covered with a watch glass to limit the evaporation of solvents present in the formula.

The options adopted for this method of measurement are as follows:

Test mode: Measurement in compression

Trigger force: 2.0 g

Pre-speed: 2.0 mm/s

Test speed 1.0 mm/s

Temperature 20° C.+/−1° C.

Penetration distance 5 mm

Three successive measurements are taken at points at least 12 mm apart, and at least 10 mm from the edge of the container. The container is held during measurement. The value adopted is the mean value of the maxima obtained in each measurement.

According to a particular embodiment of the first aspect of the invention, the texture, measured by texture analysis, counting from preparation of the composition, namely at T₀, is greater than or equal to 30 g, or 60 g or 70 g at room temperature.

Still according to one embodiment of the first aspect of the invention, the texture, measured by texture analysis, after a period of 60 days at 45° C. is less than or equal to 100 g, or even less than or equal to 90 g.

Still according to another particular embodiment of the first aspect of the invention, the change in texture measured by texture analysis in a period of 60 days at 45° C. counting from preparation of said composition, namely 24 hours after manufacture of the composition, is less than 100%, the change in texture being defined by:

$$\frac{{T}_{60 j} - {T}_{0}}{{T}_{0}} \times 100$$

where T₆₀ⱼ is the measurement from texture analysis at 60 days, and

T₀ is the measurement from texture analysis taken 24 hours after manufacture of the preparation of the composition.

According to a particular embodiment of the second, aspect, the change in texture measured by texture analysis in a period of 60 days at 45° C. counting from preparation of said composition is less than 70%, or even less than 60%, or even less than 50%, for example less than 20%, 10% or 5%.

Thus, according to its second aspect, the present invention makes it possible to prepare cosmetic compositions having a greater texture than that of the equivalent compositions currently marketed. Thus, as the increase in texture over time is less in the compositions, according to the present invention than in the compositions commonly prepared, the present invention offers the advantage that it makes it possible to prepare cosmetic compositions with a greater texture after manufacture. In other words, it is no longer necessary to anticipate as much texture development as usual, of the cosmetic compositions in question with the passage of time.

Behenyl Alcohol

Behenyl alcohol, otherwise called docosanol, is a C₂₂ fatty alcohol.

Cetyl alcohol is commonly used for thickening cosmetic compositions of the emulsion type in which the surfactant system is free from triethanolamine stearate, in particular for compositions of so-called “extending” mascaras. It is a co-surfactant of low HLB, commonly used in conjunction with a surfactant of high HLB such as potassium cetyl phosphate and/or steareth-20.

It has been observed that for cosmetic compositions of the emulsion type comprising such a surfactant system there is a tendency for their texture to vary over time, and more precisely to increase in a way that is troublesome for the applicability of the composition on the areas to be made up and/or treated.

The total or partial replacement of cetyl alcohol in cosmetic compositions of this type is precisely what is envisaged within the scope of the present invention.

Behenyl alcohol can be present at a content greater than or equal to 0.3 wt. %, in particular ≧0.5 wt. %, and more particularly ≧1 wt. %, or even ≧2 wt. % relative to the total weight of the composition.

Typically, behenyl alcohol is present at a content in the range from 0.3 to 20 wt. %, notably from 0.5 to 10 wt. %, more particularly from 0.7 to 7%, or even from 1 to 6 wt. % relative to the total weight of the composition.

Emulsifying System

The composition according to the invention is free from triethanolamine stearate. In other words, it contains less than 1 wt. % of triethanolamine stearate, preferably less than 0.1 wt. %, or even 0 wt. %, relative to the total weight of the composition.

According to the invention, generally a suitably selected emulsifying system is used for obtaining a wax-in-water or oil-in-water emulsion. In particular, the emulsifying system can comprise at least one emulsifier possessing, at 25° C., a hydrophilic-lipophilic balance (HLB), in the GRIFFIN sense, greater than or equal to 8.

The HLB value according to GRIFFIN is defined in J. Soc. Cosm. Chem. 1954 (volume 5), pages 249-256.

These emulsifiers can be selected from non-ionic, anionic, cationic, amphoteric surfactants or from polymeric surfactants. Reference may be made to the document “Encyclopedia of Chemical Technology, KIRK-OTHMER”, volume 22, p. 333-432, 3rd edition, 1979, WILEY, for definitions of the properties and (emulsifying) functions of surfactants, in particular pages 347-377 of this reference, for anionic, amphoteric and non-ionic surfactants.

The surfactants that can be used in the composition according to the invention are selected fron:

a) non-ionic surfactants with HLB greater than or equal to 8 at 25° C., used alone or mixed. We may notably mention:
esters and ethers of monosaccharides such as the mixture of cetearyl glucoside and cetyl and stearyl alcohols such as Montanov 68 from Seppic;
ethoxylated and/or propoxylated ethers (which can comprise from 1 to 150 ethoxylated and/or propoxylated groups) of glycerol;
ethoxylated and/or propoxylated ethers (which can comprise from 1 to 150 ethoxylated and/or propoxylated groups) of fatty alcohols (notably of C₈-C₂₄, and preferably C₁₂-C₁₈, alcohol) such as the ethoxylated ether of cetearyl alcohol with 30 ethoxylated groups (CTFA name “Ceteareth-30”), the ethoxylated ether of stearyl alcohol with 20 ethoxylated groups (CTFA name “Steareth-20”), the ethoxylated ether of the mixture of C₁₂-C₁₅ fatty alcohols having 7 ethoxylated groups (CTFA name “C₁₂₋₁₅ Pareth-7”) notably marketed under the designation NEODOL 25-7® by SHELL CHEMICALS
esters of fatty acid (notably of C₈-C₂₄, and preferably C₁₆-C₂₂, acid) and of polyethylene glycol (which can comprise from 1 to 150 ethylene glycol units) such as the stearate of PEG-50 and the monostearate of PEG-40 notably marketed under the name MYRJ 52P® by the company ICI UNIQUEMA, or PEG-30 glyceryl stearate notably marketed under the name TAGAT S® by the company Evonik GOLDSCHMIDT;
esters of fatty acid (notably of C₈-C₂₄, and preferably C₁₆-C₂₂, acid) and ethoxylated and/or propoxylated glycerol ethers (which can comprise from 1 to 150 ethoxylated and/or propoxylated groups), such as PEG-200 glyceryl monostearate notably sold under the designation Simulsol 220 TM® by the company SEPPIC; polyethoxylated glyceryl stearate with 30 ethylene oxide groups such as the product TAGAT S® sold by the company Evonik GOLDSCHMIDT, polyethoxylated glyceryl oleate with 30 ethylene oxide groups such as the product TAGAT O® sold by the company Evonik GOLDSCHMIDT, polyethoxylated glyceryl cocoate with 30 ethylene oxide groups such as the product VARIONIC LI 13® sold by the company SHEREX, polyethoxylated glyceryl isostearate with 30 ethylene oxide groups such as the product TAGAT L® sold by the company Evonik GOLDSCHMIDT and polyethoxylated glyceryl laurate with 30 ethylene oxide groups such as the product TAGAT I® from the company Evonik GOLDSCHMIDT,
esters of fatty acid (notably of C₈-C₂₄, and preferably C₁₆-C₂₂, acid) and ethoxylated and/or propoxylated sorbitol ethers (which can comprise from 1 to 150 ethoxylated and/or propoxylated groups), such as polysorbate 20 notably sold under the designation Tween 20® by the company CRODA, polysorbate 60 notably sold under the designation Tween 60® by the company CRODA,
dimethicone copolyol, such as that sold under the designation Q2-5220® by the company DOW CORNING,
dimethicone copolyol benzoate (FINSOLV SLB 101® and 201® from the company FINTEX),
copolymers of propylene oxide and ethylene oxide, also called EO/PO polycondensates,
and mixtures thereof.

The EO/PO polycondensates are more particularly copolymers consisting of polyethylene glycol and polypropylene glycol blocks, for example polyethylene glycol/polypropylene glycol/polyethylene glycol triblock polycondensates. These triblock polycondensates have for example the following chemical structure:

H—(O—CH₂—CH₂)ₙ—(O—CH(CH₃)—CH₂)_{b}—(O—CH₂—CH₂)ₐ—OH,

where a is from 2 to 120, and b is from 1 to 100.

The EO/PO polycondensate preferably has a weight-average molecular weight in the range from 1000 to 15000, and more preferably in the range from 2000 to 13000. Advantageously, said EO/PO polycondensate has a cloud point, at 10 g/l in distilled water, greater than or equal to 20° C., preferably greater than or equal to 60° C. The cloud point is measured according to standard ISO 1065. As EO/PO polycondensate usable according to the invention, we may mention the polyethylene glycol/polypropylene glycol/polyethylene glycol triblock polycondensates sold under the designations SYNPERONIC® such as SYNPERONIC PE/L44® and SYNPERONIC PE/F127® by the company ICI.

b) non-ionic surfactants with HLB less than 8 at 25° C., optionally combined with one or more non-ionic surfactants with HLB greater than 8 at 25° C. such as those mentioned above; we may notably mention:
esters and ethers of monosaccharides such as sucrose stearate, sucrose cocoate, sorbitan stearate and mixtures thereof such as Arlatone 2121® marketed by the company ICI;
ethoxylated and/or propoxylated ethers (which can comprise from 1 to 150 ethoxylated and/or propoxylated groups) of fatty alcohols (notably of C₈-C₂₄, and preferably C₁₂-C₁₈, alcohol) such as the ethoxylated ether of stearyl alcohol with 2 ethoxylated groups (CTFA name “Steareth-2”);
esters of fatty acids (notably of C₈-C₂₄, and preferably C₁₆-C₂₂, acid) and of polyol, notably of glycerol or of sorbitol, such as glyceryl stearate, such as the product sold under the designation TEGIN M® by the company Evonik GOLDSCHMIDT, glyceryl laurate such as the product sold under the designation IMWITOR312® by the company HULS, polyglyceryl-2 stearate, sorbitan tristearate, glyceryl ricinoleate;
lecithins, such as soya lecithins (such as Emulmetik 100 J from Cargill, or Biophilic H from, Lucas Meyer);
the mixture of cyclomethicone/dimethicone copolyol sold under the designation Q2-3225C® by the company DOW CORNING.

c) anionic surfactants such as:
polyethoxylated salts of fatty acids notably those derived from amines or alkali metal salts, and mixtures thereof;
phosphoric esters and salts thereof such as “DEA oleth-10 phosphate” (Crodafos N 10N from the company CRODA) or monopotassium monocetyl phosphate or potassium cetyl phosphate (Amphisol K from Givaudan);
sulphosuccinates such as “Disodium PEG-5 citrate lauryl sulphosuccinate” and “Disodium ricinoleamido MEA sulphosuccinate”;
alkyl ethersulphates such as sodium lauryl ether sulphate;
isethionates;
acylglutamates such as “Disodium hydrogenated tallow glutamate” (AMISOFT HS-21 R® marketed by the company AJINOMOTO) and sodium stearoyl glutamate (AMISOFT HS-11 PF® marketed by the company AJINOMOTO) and mixtures thereof;
derivatives of soya such as potassium soyate;
citrates, such as glyceryl stearate citrate (Axol C 62 Pellets from Degussa);
derivatives of proline, such as sodium palmitoyl proline (Sepicalm VG from Seppic), or the mixture of sodium palmitoyl sarcosinate, magnesium palmitoyl glutamate, palmitic acid and palmitoyl proline (Sepifeel One from Seppic);
lactylates, such as sodium stearoyl lactylate (Akoline SL from Karlshamns AB);
sarcosinates, such as sodium, palmitoyl sarcosinate (Nikkol sarcosinate PN) or the mixture of stearoyl sarcosine and myristoyl sarcosine 75125 (Crodasin SM from Croda);
sulphonates, such as sodium C₁₄₋₁₇ alkyl sec sulphonate (Hostapur SAS 60 from Clariant);
glycinates, such as sodium cocoyl glycinate (Amilite GCS-12 from Ajinomoto).

The compositions according to the invention can also contain one or more amphoteric surfactants such as betaines or N-acyl-amino acids such as the N-alkyl-amino acetates and disodium cocoamphodiacetate and the amine oxides such as stearamine oxide or silicone surfactants such as the dimethicone copolyol phosphates such as that sold under the designation PECOSIL PS 100® by the company PHOENIX CHEMICAL.

The emulsifier that can be used can also be a polymeric surfactant, notably a thermogelling polymer.

According to a particular embodiment, the emulsifying system comprises at least one surfactant selected from:
i) an alkali metal alkyl phosphate or phosphine oxide of formula (R—O)ₙ—P═O—(O⁻M)ₘ with R representing a linear or branched C₈-C₂₂ alkyl group such as cetyl, n being equal to 1, 2 or 3 and in being equal to 0, 1 or 2, with m+n being equal to 3 and M representing a hydrogen atom or an alkali metal or alkaline-earth metal, preferably n=1 and m=2, and M is an alkali metal, such as sodium or potassium,
ii) a polyethoxylated alcohol of formula R′—(OCH₂CH₂)ₚ—OH with R′ representing a linear or branched C₁-C₃₀ alkyl and in particular represents CH₃—(CH₂)₁₇— and with p representing an integer between 1 and 30 inclusive, preferably between 2 and 20; such as steareth-20 and steareth-2,
iii) a salt of glutamic acid of formula R—CONH—C(COO⁻M)-C₂H₄—COO-M′ with R representing a linear or branched C₈-C₂₂ alkyl group such as stearyl and M′ representing an alkali metal or alkaline-earth metal, and
iv) an alkyl glucoside obtained by condensation of glucose and of linear or branched C₈-C₂₂ fatty alcohols such as a cetyl and stearyl mixture called cetearyl.

As an example of a surfactant according to point (i) above, we may mention potassium cetyl phosphate notably sold under the name Amphisol K by the company Givaudan.

As an example of a surfactant according to point (iii) above we may mention sodium stearoyl glutamate and as a surfactant according to point (iv) above we may mention cetearyl glucoside.

According to an even more particular embodiment, the emulsifying system comprises at least one of these two surfactants or mixture thereof.

According to another particular embodiment of the invention, the emulsifying system comprises at least one emulsifier selected from (a) ethoxylated and/or propoxylated ethers (which can comprise from 1 to 150 ethoxylated and/or propoxylated groups) of fatty alcohols (notably of C₈-C₂₄, and preferably C₁₂-C₁₈, alcohol) such as the ethoxylated ether of stearyl alcohol with 2 ethoxylated groups (CTFA name “Steareth-2”); (b) esters of fatty acids (notably of C₈-C₂₄, and preferably C₁₆-C₂₂, acid) and of polyol, notably of glycerol or of sorbitol, such as glyceryl stearate such as the product sold under the designation TEGIN M® by the company Evonik GOLDSCHMIDT; (c) esters of phosphoric acid and alkali metal salts thereof such as potassium cetyl phosphate (Amphisol K from Givaudan) and/or (d) esters of fatty acid (notably of C₈-C₂₄, and preferably C₁₆-C₂₂, acid) and of ethoxylated and/or propoxylated glycerol ethers (which can comprise from 1 to 150 ethoxylated and/or propoxylated groups), such as polyethoxylated glyceryl stearate with 30 ethylene oxide groups such as the product TAGAT® S sold by the company Evonik GOLDSCHMIDT and (e) mixtures thereof.

According to a particular embodiment of the invention, the emulsifying system comprises at least one ethoxylated and/or propoxylated ether (which can comprise from 1 to 150 ethoxylated and/or propoxylated groups) of fatty alcohols (notably of C₈-C₂₄, and preferably C₁₂-C₁₈, alcohol) such as the ethoxylated ether of stearyl alcohol with 2 ethoxylated groups (CTFA name. “Steareth-2”), and at least one alkali metal alkyl phosphate or phosphine oxide of formula (R—O)ₙ—P═O—(O⁻M)ₘ with R representing a linear or branched C₈-C₂₂ alkyl group such as cetyl, n being equal to 1, 2 or 3 and m being equal to 0, 1 or 2, with m+n being equal to 3 and M representing a hydrogen atom or an alkali metal or alkaline-earth metal, preferably n=1 and m=2, and M is an alkali metal, such as sodium or potassium, as surfactants.

According to a particular embodiment, the emulsifying system comprises at least one phosphate surfactant, notably potassium cetyl phosphate.

According to a particular embodiment, the emulsifying system comprises at least one surfactant selected from steareth-2, glyceryl stearate, polyethoxylated glyceryl stearate with 30 ethylene oxide groups, potassium cetyl phosphate or mixtures thereof.

According to one embodiment, the emulsifying system of the composition according to the invention comprises at least one emulsifier selected from potassium cetyl phosphate, steareth-2, steareth-20 and mixture thereof.

According to one embodiment, the emulsifying system of the composition according to the invention comprises potassium cetyl phosphate and steareth-2.

According to another embodiment, the emulsifying system comprises a surfactant with HLB greater than 8 together with a surfactant with HLB less than 8.

According to one embodiment, the composition according to the invention comprises at least one anionic surfactant and at feast one non-ionic surfactant, in particular a non-ionic surfactant with HLB less than or equal to 8 to 25° C., and said surfactants can advantageously be selected from the surfactants mentioned above.

The composition according to the invention can contain from 0.01 to 30 wt. % of emulsifier, relative to the total weight of said composition, preferably from 1 to 15 wt. % and more preferably from 2 to 13 wt. %.

According to another embodiment, the composition according to the invention comprises at least one emulsifier selected from esters of fatty acids and polyol, notably glyceryl stearate, esters of fatty acid and polyethylene glycol, notably PEG-30 stearate, and mixtures thereof.

Co-Surfactants

According to a particular embodiment, the compositions according to the invention can further comprise at least one co-surfactant other than behenyl alcohol.

The co-surfactants can notably be selected from fatty alcohols, preferably comprising from 10 to 30 carbon atoms. “Fatty alcohol comprising 10 to 30 carbon atoms” means any pure saturated or unsaturated, linear or branched fatty alcohol, having from 10 to 30 carbon atoms.

As examples of fatty alcohols that can be used in conjunction with the emulsifier(s) of the emulsifying system according to the invention, we may mention linear or branched fatty alcohols, of synthetic or natural origin, for example alcohols derived from vegetable materials (copra, cabbage palm, palm etc.) or animal (tallow etc.). Of course, other long-chain alcohols can also be used, for example ether alcohols or the so-called Guerbet alcohols. Finally, it is also possible to use certain fractions of varying length from alcohols of natural origin, for example coco (C₁₂ to C₁₆) or tallow (C₁₆ to C₁₈) or compounds such as diols or cholesterol.

It is preferable to use a fatty alcohol comprising 10 to 26 carbon atoms, preferably from 10 to 24 carbon atoms, and more preferably from 12 to 21 carbon atoms.

As particular examples of fatty alcohols usable within the scope of the present invention, we may notably mention lauric; myristic, cetyl, stearyl, isostearyl, palmitic, oleic, cetearyl (mixture of cetyl and stearyl alcohol), erucic, arachidyl alcohol and mixtures thereof.

Said fatty alcohols are notably marketed under the designation NAFOL by the company SASOL.

Among the co-surfactants usable according to the invention, we may also mention glyceryl mono- and/or distearate.

The co-surfactant or co-surfactants can be present at a content in the range from 0.05 to 15 wt. %, preferably from 0.1 to 10 wt. %, and more preferably from 1 to 8 wt. % relative to the total weight of the composition.

Preferred Emulsifying Systems

According to a particular embodiment of the invention, the emulsifying system of the composition according to the invention comprises at least one surfactant according to point (i) mentioned previously and/or at least one surfactant according to point (iii) also mentioned above, as well as optionally at least one surfactant according to point (ii) and/or at least one surfactant according to point (iv) and/or at least one fatty alcohol comprising from 10 to 26 carbon atoms, preferably from 10 to 24 carbon atoms, and more preferably from 12 to 21 carbon atoms,

In other words, the following embodiments are particularly in keeping with the invention.

According to a particular embodiment, said emulsifier is an alkali metal alkyl phosphate or phosphine oxide of formula (R—O)ₙ—P═O—(O⁻M)ₘ with R representing a linear or branched C₈-C₂₂ alkyl group such as cetyl, n being equal to 1, 2 or 3 and m being equal to 0, 1 or 2, with m+n being equal to 3 and M representing a hydrogen atom or an alkali metal or alkaline-earth metal, preferably n=1 and m=2, and M is an alkali metal, such as sodium or potassium.

According to this embodiment, said emulsifier is preferably potassium cetyl phosphate.

According to another particular embodiment, the emulsifying system of the composition according to the invention can also comprise, in addition to an alkali metal alkyl phosphate or phosphine oxide described above, an ethoxylated and/or propoxylated ether (which can comprise from 1 to 150 ethoxylated and/or propoxylated groups) of fatty alcohols (notably of C₈-C₂₄, and preferably C₁₂-C₁₈ alcohol) such as ethoxylated ether of stearyl alcohol with 2 ethoxylated groups (CTFA name “Steareth-2”).

According to this embodiment, the emulsifying system of the composition according to the invention preferably comprises potassium cetyl phosphate and Steareth-2.

According to a particular embodiment, said emulsifier is a salt of glutamic acid of formula R—CONH—C(COO⁻M)-C₂H₄—COO-M′ with R representing a linear or branched C₈-C₂₂ alkyl group such as stearyl and M′ representing an alkali metal or alkaline-earth metal.

According to this embodiment, said emulsifier is preferably sodium stearoyl glutamate.

According to another particular embodiment, the emulsifying system of the composition according to the invention can also comprise, in addition to a salt of glutamic acid described above, an alkyl glucoside obtained by condensation of glucose and of linear or branched C₈-C₂₂ fatty alcohols such as a cetyl and stearyl mixture, called cetearyl.

According to this embodiment, the emulsifying system of the composition according to the invention preferably comprises sodium stearoyl glutamate and cetearyl glucoside.

According to another particular embodiment, the emulsifying system of the composition according to the invention can comprise a salt of glutamic acid described above and an alkali metal alkyl phosphate or phosphine oxide also described above.

According to this embodiment, the emulsifying system of the composition according to the invention preferably comprises sodium stearoyl glutamate and potassium cetyl phosphate.

According to another particular embodiment, the emulsifying system of the composition according to the invention can comprise a salt of glutamic acid, an alkali metal alkyl phosphate or phosphine oxide and an ethoxylated and/or propoxylated ether of fatty alcohols as described above.

According to this embodiment, the emulsifying system of the composition according to the invention preferably comprises sodium stearoyl glutamate, potassium cetyl phosphate and steareth-2.

According to a particular embodiment, the emulsifying system of the composition according to the invention comprises, in addition to an alkali metal alkyl phosphate or phosphine oxide described above, a co-surfactant selected from fatty alcohols comprising from 10 to 26 carbon atoms, preferably from 10 to 24 carbon atoms, and more preferably from 12 to 21 carbon atoms.

According to this embodiment, the emulsifying system of the composition according to the invention preferably comprises potassium cetyl phosphate and cetyl alcohol.

According to a particular embodiment, the emulsifying system of the composition further comprises at least one ethoxylated and/or propoxylated ether of fatty alcohols as described above.

According to this embodiment, the emulsifying system of the composition according to the invention preferably comprises potassium cetyl phosphate, steareth-2 and cetyl alcohol.

According to this same embodiment, the emulsifying system of the composition according to the invention preferably comprises potassium cetyl phosphate, steareth-2, steareth-20 and cetyl alcohol.

According to a particular embodiment, the emulsifying system of the composition according to the invention comprises a salt of glutamic acid of formula R—CONH—C(COO⁻M)-C₂H₄—COO-M′ with R representing a linear or branched C₈-C₂₂ alkyl group such as stearyl and M′ representing an alkali metal or alkaline-earth metal and a co-surfactant selected from fatty alcohols comprising from 10 to 26 carbon atoms, preferably from 10 to 24 carbon atoms, and more preferably from 12 to 21 carbon atoms.

According to this preferred embodiment, the emulsifying system of the composition according to the invention preferably comprises sodium stearoyl glutamate and cetyl alcohol.

According to a particular embodiment, the emulsifying system of the composition further comprises an alkyl glucoside obtained by condensation of glucose and of linear or branched C₈-C₂₂ fatty alcohols such as a cetyl and stearyl mixture called cetearyl.

According to this embodiment, the emulsifying system of the composition according to the invention preferably comprises sodium stearoyl glutamate, cetearyl glucoside and cetyl alcohol.

Finally, according to a particular embodiment, the emulsifying system of the composition further comprises an alkali metal alkyl phosphate or phosphine oxide of formula (R—O)ₙ—P═O—(O⁻M)ₘ with R representing a linear or branched C₈-C₂₂ alkyl group such as cetyl, n being equal to 1, 2 or 3 and in being equal to 0, 1 or 2, with m+n being equal to 3 and M representing a hydrogen atom or an alkali metal or alkaline-earth metal, preferably n=1 and m=2, and M is an alkali metal, such as sodium or potassium.

According to this embodiment, the emulsifying system of the composition according to the invention preferably comprises sodium stearoyl glutamate, potassium cetyl phosphate and cetyl alcohol.

Physiologically Acceptable Medium

The compositions according to the invention can comprise a physiologically acceptable medium, i.e. a medium that is non-toxic and that can be applied on the keratin fibres of human beings and has a pleasant appearance, odour and feel.

The physiologically acceptable medium generally has to be suited to the nature of the substrate on which the composition is to be applied, as well as the form in which the composition is to be packaged.

The compositions according to the invention can be in the form of emulsion obtained by dispersing a fatty phase in an aqueous phase, either directly or indirectly.

They can be single emulsions obtained by dispersing a fatty phase in an aqueous phase (O/W), or an emulsion of the multiple emulsion type:

According to a particular embodiment, the composition of the invention is in the form of a wax-in-water or oil-in-water emulsion.

The compositions of the invention can be of liquid or semi-liquid consistency of the milk type, or of soft, semi-solid or solid consistency of the cream or gel type.

These compositions are prepared according to the usual methods.

### Aqueous Phase

The composition according to the invention can comprise an aqueous phase, which forms the continuous phase.

“Composition with aqueous continuous phase” means that the composition has a conductivity, measured at 25° C., greater than or equal to 23 μS/cm (microsiemens/cm), the conductivity being measured for example by means of an MPC227 conductivity meter from Mettler Toledo and an Inlab730 conductivity measurement cell. The measurement cell is immersed in the composition, in such a way as to eliminate the air bubbles that may form between the 2 electrodes of the cell. The conductivity reading is taken once the value indicated by the conductivity meter has stabilized. The mean value is calculated from at least 3 successive measurements.

The aqueous phase comprises water and/or at least one water-soluble solvent.

“Water-soluble solvent” means, in the present invention, a compound that is liquid at room temperature and is miscible with water (water miscibility greater than 50 wt. % at 25° C. and atmospheric pressure).

The water-soluble solvents usable in the compositions according to the invention can moreover be volatile.

Among the water-soluble solvents that can be used in the compositions according to the invention, we may notably mention monohydric lower alcohols having from 1 to 5 carbon atoms such as ethanol and isopropanol, glycols having from 2 to 8 carbon atoms such as ethylene glycol, propylene glycol, 1,3-butylene glycol and dipropylene glycol, C₃-C₄ ketones and C₂-C₄ aldehydes.

The aqueous phase (water and optionally the water-miscible solvent) is generally present in the composition according to the present application at a content in the range from 1 to 80 wt. %, relative to the total weight of the composition, preferably in the range from 10 to 70 wt. %, preferably in the range from 15 to 60 wt. %, and more preferably from 30 to 60 wt. %.

Fatty Phase

The composition according to the invention can comprise at least one liquid and/or solid fatty phase. This fatty phase can comprise at least one wax, pasty fat, oil or mixture thereof.

The fatty phase can be present in a composition according to the invention at a content in the range from 1 to 70 wt. %, relative to the total weight of the composition, preferably in the range from 2 to 50 wt. %, preferably in the range from 5 to 40 wt. %, more preferably in the range from 15 to 40 wt. %.

According to a preferred embodiment, a composition according to the invention further comprises at least one lipophilic structure-forming agent such as waxes, pasty fats and mixtures thereof.

Waxes

The compositions according to the invention can optionally comprise at least one wax or mixture of waxes. These waxes can be solid at room temperature and at atmospheric pressure.

The compositions according to the invention, such as mascaras, can comprise one or more waxes, at a content in the range from 1 to 60 wt. % relative to the total weight of the composition, notably from 2 to 45 wt. %, preferably in the range from 15 to 40 wt. %.

According to a particular embodiment, a composition of the invention can notably be in the form of a wax-in-water emulsion, in other words can comprise a dispersion of a wax or mixture of waxes in an aqueous continuous phase.

The wax considered within the scope of the present invention is generally a lipophilic compound, solid at room temperature (25° C.), with reversible solid/liquid change of state, having a melting point greater than or equal to 30° C. and up to 120° C., with the exception of the fatty alcohols, such as described previously, notably fatty alcohols having from 10 to 30 carbon atoms and notably from 12 to 22 carbon atoms.

By bringing the wax to the liquid state (melting), it is possible to make it miscible with oils and to form a microscopically homogeneous mixture, but when the temperature of the mixture returns to room temperature there is recrystallization of the wax in the oils of the mixture.

In particular, waxes suitable for the invention can have a melting point above about 45° C., and in particular above 55° C.

The melting point of the wax can be measured by means of a differential scanning calorimeter (DSC), for example the calorimeter sold under the designation DSC 30 by the company METLER.

The measurement protocol is as follows:

A 15 mg sample of product in a crucible is submitted to a first temperature rise from 0° C. to 120° C., at a heating rate of 10° C./minute, then it is cooled from 120° C. to 0° C. at a cooling rate of 10° C./minute and is finally submitted to a second temperature rise from 0° C. to 120° C. at a heating rate of 5° C./minute. During the second temperature rise, the variation of the difference in power absorbed by the empty crucible and by the crucible containing the sample of product is measured as a function of temperature. The melting point of the compound is the temperature value corresponding to the top of the peak of the curve representing the variation of the difference in power absorbed as a function of temperature.

The waxes that can be used in the compositions according to the invention are selected from solid waxes, deformable or not at room temperature, of animal, vegetable, mineral or synthetic origin and mixtures thereof.

The wax can also have a hardness in the range from 0.05 MPa to 30 MPa, and preferably in the range from 6 MPa to 15 MPa. The hardness is determined by measuring the compressive force measured at 20° C. by means of the texture analyser sold under the designation TA-TX2i by the company RHEO, equipped with a stainless steel spindle with a diameter of 2 mm moving at a speed of measurement of 0.1 minis, and penetrating into the wax to a depth of penetration of 0.3 mm.

The measurement protocol is as follows:

The wax is melted at a temperature equal to the melting point of the wax+20° C. The molten wax is cast in a container 30 mm in diameter and 20 mm deep. The wax is recrystallized at room temperature (25° C.) for 24 hours, then the wax is stored for at least 1 hour at 20° C. before measuring the hardness. The hardness value is the maximum compressive force measured divided by the area of the spindle of the texture analyser in contact with the wax.

It is notably possible to use hydrocarbon waxes such as beeswax, lanolin wax, and Chinese insect wax; rice wax, carnauba wax, candelilla wax, ouricury wax; alfa wax, cork fibre wax, sugarcane wax, Japan wax and sumach wax; montan wax, microcrystalline waxes, paraffins and ozokerite; polyethylene waxes, waxes obtained by Fischer-Tropsch synthesis and waxy copolymers and their esters.

We may also mention the waxes obtained by catalytic hydrogenation of animal or vegetable oils having linear or branched C₈-C₃₂ fatty chains.

Among the latter, we may notably mention hydrogenated jojoba oil, hydrogenated sunflower oil, hydrogenated castor oil, hydrogenated copra oil and hydrogenated lanolin oil, di-(trimethylol-1,1,1-propane) tetrastearate sold under the designation “HEST 2T-4S” by the company HETERENE, di-(trimethylol-1,1,1-propane) tetrabehenate sold under the designation HEST 2T-4B by the company HETERENE.

It is also possible to use waxes obtained by transesterification and hydrogenation of vegetable oils, such as castor oil or olive oil, such as the waxes sold under the designations Phytowax ricin 16L64® and 22L73® and Phytowax Olive 18L57 by the company SOPHIM. These waxes are described in application FR-A-2792190.

It is also possible to use silicone waxes, which can advantageously be substituted polysiloxanes, preferably of low melting point. These are notably substituted linear polysiloxanes constituted essentially (apart from the end groups) of units of formulae II and III, in the respective molar proportions m and n:

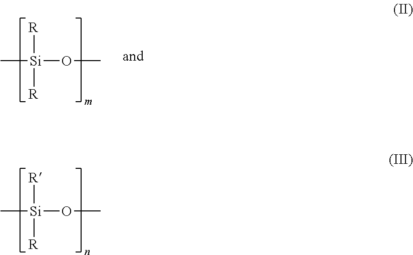

in which:

each substituent R is defined as previously,

each R′ represents independently an alkyl (linear or branched), optionally unsaturated, having 6-30 carbon atoms, or else a group —X—R″, each X representing independently:

—O—,

—(CH₂)ₐ—O—CO—,

—(CH₂)_{b}—CO—O—,

a and b represent independently numbers in the range from 0 to 6, and

each R″ represents independently an alkyl group, optionally unsaturated, having 6 to 30 carbon atoms,
m is a number in the range from 0 to 400, and in particular from 0 to 100,
n is a number in the range from 1 to 200, and in particular from 1 to 100,

the sum (m+n) being less than 400, and in particular less than or equal to 100.

These silicone waxes are known or can be prepared according to known methods. Among the commercial silicone waxes of this type, we may notably mention those sold under the designations Abilwax 9800, 9801 or 9810 (GOLDSCHMIDT), KF910 and KF7002 (SHIN ETSU), or 176-1118-3 and 176-11481 (GENERAL ELECTRIC).

The silicone waxes that can be used can also be selected from the compounds of the following formula (IV):

R₁—Si(CH₃)₂—O—[Si(R)₂—O—]z—Si(CH₃)₂—R₂ (IV)

in which:

R is defined as previously,

R₁ represents an alkyl group having from 1 to 30 carbon atoms, an alkoxy group having from 6 to 30 carbon atoms, or a group of formula:

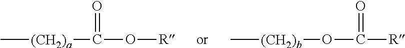

R₂ represents an alkyl group with 6 to 30 carbon atoms, an alkoxy group having from 6 to 30 carbon atoms or a group of formula:

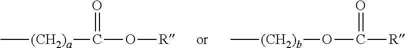

a and b representing a number from 0 to 6,
R″ being an alkyl having from 6 to 30 carbon atoms,
and z is a number in the range from 1 to 100.

Among the silicone waxes of formula (IV), we may notably mention the alkyl or alkoxydimethicones such as the following commercial products: Abilwax 2428, 2434 and 2440 (GOLDSCHMIDT), or VP 1622 and VP 1621 (WACKER), as well as (C₂₀-C₆₀) alkyldimethicones, in particular the (C₃₀-C₄₅) alkyldimethicones such as the silicone wax sold under the designation SF-1642 by the company GE-Bayer Silicones.

It is also possible to use hydrocarbon waxes modified with silicone or fluorine containing groups, for example: siliconyl candelilla, siliconyl beeswax and Fluorobeeswax from Koster Keunen.

The waxes can also be selected from the fluorinated waxes.

According to a particular embodiment, the compositions according to the invention can comprise at least one so-called sticky wax, i.e. possessing tack greater than or equal to 0.7 N.s and a hardness less than or equal to 3.5 MPa.

The use of a sticky wax can notably provide a cosmetic composition that can be applied easily on the eyelashes, having good adherence on the eyelashes and which leads to the formation of smooth, homogeneous and thickening make-up.

The sticky wax used can notably possess tack in the range from 0.7 N.s to 30 N.s, in particular greater than or equal to 1 N.s, notably in the range from 1 N.s to 20 N.s, in particular greater than or equal to 2 N.s, notably in the range from 2 N.s to 10 N.s, and in particular in the range from 2 N.s to 5 N.s.

The tack of a wax is determined by measuring the variation of force (compressive force or pulling force) as a function of time, at 20° C. by means of the texture analyser sold under the designation “TA-TX2i®” by the company RHEO, equipped with a spindle made of acrylic polymer of conical shape forming an angle of 45°.

The measurement protocol is as follows:

The wax is melted at a temperature equal to the melting point of the wax+10° C. The molten wax is cast in a container 25 min in diameter and 20 mm deep. The wax is recrystallized at room temperature (25° C.) for 24 hours in such a way that the surface of the wax is flat and smooth, then the wax is stored for at least 1 hour at 20° C. before measuring the tack.

The spindle of the texture analyser is moved at a speed of 0.5 mm/s, then it penetrates into the wax to a depth of penetration of 2 mm. When the spindle has penetrated into the wax to the depth of 2 mm, the spindle is kept still for 1 second (corresponding to the relaxation time) and then is retracted at a speed of 0.5 mm/s.

During the relaxation time, the force (compressive force) decreases rapidly to zero, then, during retraction of the spindle, the force (pulling force) becomes negative and then increases again to the value 0. The tack, corresponds to the integral of the curve of the force as a function of time for the portion of the curve corresponding to the negative values of the force (pulling force): The value of the tack is expressed in N.s.

The sticky wax that can be used generally has a hardness less than or equal to 3.5 MPa, in particular in the range from 0.01 MPa to 3.5 MPa, notably in the range from 0.05 MPa to 3 MPa, or especially in the range from 0.1 MPa to 2.5 MPa.

The hardness is measured according to the protocol described previously.

The sticky wax used can be a C₂₀-C₄₀ alkyl (hydroxystearyloxy)stearate (with the alkyl group comprising 20 to 40 carbon atoms), alone or mixed, in particular a C₂₀-C₄₀ alkyl 12-(12′-hydroxystearyloxy)stearate.

Such a wax is notably sold under the designations “Kester Wax K 82 P®” and “Kester Wax K 80 P®” by the company KOSTER KEUNEN.

The aforementioned waxes generally have an initial melting point below 45° C.

The wax or waxes can be present in the form of an aqueous microdispersion of wax. “Aqueous microdispersion, of wax” means an aqueous dispersion of wax particles, in which the size, expressed as “effective” mean diameter by volume D[4.3], of said wax particles is less than or equal to about 1 μm.

The microdispersions of wax are stable dispersions of colloidal particles of wax, and are notably described in “Microemulsions Theory and Practice”, L. M. Prince Ed., Academic Press (1977) pages 21-32.

In particular, these microdispersions of wax can be obtained by melting the wax in the presence of a surfactant, and optionally a portion of the water, then gradually adding hot water with stirring. Intermediate formation of an emulsion of the water-in-oil type is observed, followed by phase inversion, finally obtaining a microemulsion of the oil-in-water type. On cooling, a stable microdispersion of colloidal solid particles of wax is obtained.

The microdispersions of wax can also be obtained by agitation of a mixture of wax, surfactant and water by means of agitating means such as ultrasound, a high-pressure homogenizer, and turbines.

The particles of the microdispersion of wax preferably have average size less than 1 μm (notably in the range from 0.02 μm to 0.99 μm), preferably less than 0.5 μm (notably in the range from 0.06 μm to 0.5 μm).

These particles are constituted essentially of a wax or of a mixture of waxes. They can, however, include a small proportion of oily and/or pasty fatty additives, a surfactant and/or a usual fat-soluble additive/active ingredient.

Pasty Compounds

The compositions according to the invention, in particular the compositions of the mascara type, can further comprise at least one pasty compound.

“Pasty compound” in the sense of the present invention means a lipophilic fatty compound with reversible solid/liquid change of state and having a liquid fraction and a solid fraction at a temperature of 23° C.

In other words, the initial melting point of the pasty compound is below 23° C. The liquid fraction of the pasty compound, measured at 23° C., represents from 20 to 97 wt. % of the pasty compound. This liquid fraction at 23° C. more preferably represents from 25 to 85%, and even more preferably from 30 to 60 wt. % of the pasty compound.

The liquid fraction by weight of the pasty compound at 23° C. is equal to the ratio of the enthalpy of fusion consumed at 23° C. to the enthalpy of fusion of the pasty compound.

The enthalpy of fusion consumed at 23° C. is the amount of energy absorbed by the sample for changing from the solid state to the state that it has at 23° C. constituted of a liquid fraction and a solid fraction.

The enthalpy of fusion of the pasty compound is the enthalpy consumed by the compound for transition from the solid state to the liquid state. The pasty compound is said to be in the solid state when the whole of its mass is in the solid form. The pasty compound is said to be in the liquid state when the whole of its mass is in the liquid form.

The enthalpy of fusion of the pasty compound is equal to the area under the curve of the thermogram obtained using a differential scanning calorimeter (DSC), such as the calorimeter sold under the designation MDSC 2920 by the company TA instrument, with a temperature rise of 5 or 10° C. per minute, according to standard ISO 11357-3:1999. The enthalpy of fusion of the pasty compound is the amount of energy necessary for transition of the compound from the solid state to the liquid state. It is expressed in J/g.

The liquid fraction of the pasty compound, measured at 32° C., preferably represents from 40 to 100 wt. % of the pasty compound, more preferably from 50 to 100 wt. % of the pasty compound. When the liquid fraction of the pasty compound measured at 32° C. is equal to 100%, the temperature of the end of the melting range of the pasty compound is less than or equal to 32° C.

The liquid fraction of the pasty compound, measured at 32° C., is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

The pasty compound preferably has a hardness at 20° C. in the range from 0.001 to 0.5 MPa, preferably from 0.002 to 0.4 MPa.

The hardness is measured by a method of penetration of a probe into a sample of the compound and in particular by means, of a texture analyser (for example TA-XT2i from Rheo) equipped with a stainless steel cylindrical spindle with a diameter of 2 mm. The hardness is measured at 20° C. at the centre of 5 samples. The spindle is introduced into each sample, the depth of penetration being 0.3 mm. The value found for the hardness is that of the maximum peak.

The pasty compound cane be selected from synthetic compounds and compounds of vegetable origin. A pasty compound can be obtained by synthesis from starting products of vegetable origin.

The pasty compound is advantageously selected from:
lanolin and its derivatives such as lanolin alcohol, ethoxylated lanolins, acetylated lanolin, lanolin esters such as isopropyl lanolate, propoxylated lanolins,
polymeric or non-polymeric silicone compounds such as polydimethysiloxanes of high molecular weights, polydimethysiloxanes with side chains of the alkyl or alkoxy type having from 8 to 24 carbon atoms, notably stearyl dimethicones,
polymeric or non-polymeric fluorinated compounds,
vinylic polymers, notably
homopolymers of olefins,
copolymers of olefins,
homopolymers and copolymers of hydrogenated dienes,
linear or branched oligomers, homo- or copolymers of alkyl (meth)acrylates preferably having a C₈-C₃₀ alkyl group,
oligomers, homo- and copolymers of vinyl esters having C₈-C₃₀ alkyl groups,
oligomers, homo- and copolymers of vinyl ethers having C₈-C₃₀ alkyl groups,
fat-soluble polyethers resulting from polyetherification between one or more C₂-C₁₀₀, preferably C₂-C₅₀, diols,
esters and polyesters,
and mixtures thereof.

The pasty compound can be a polymer, notably hydrocarbon.

A preferred silicone and fluorinated pasty, compound is polymethyltrifluoropropylmethylalkyldimethylsiloxane, manufactured under the designation X22-1088 by SHIN ETSU.

When the pasty compound is a silicone and/or fluorinated polymer, the composition advantageously comprises a compatibilizing agent such as the short-chain esters such as isodecyl neopentanoate.

Among the fat-soluble polyethers, we may notably mention the copolymers of ethylene oxide and/or of propylene oxide with C₆-C₃₀ alkylene oxides. Preferably, the weight ratio of ethylene oxide and/or propylene oxide to the alkylene oxides in the copolymer is from 5:95 to 70:30. In this class, we may notably mention the block copolymers comprising blocks of C₆-C₃₀ alkylene oxides having a molecular weight in the range from 1000 to 10000, for example a polyoxyethylene/polydodecylene glycol block copolymer such as the ethers of dodecanediol (22 mol) and of polyethylene glycol (45 oxyethylene units or OE) marketed under the brand name ELFACOS ST9 by Akzo Nobel.

Among the esters, the following are notably preferred:
esters of an oligomeric glycerol, notably the diglycerol esters, in particular condensates of adipic acid and glycerol, for which a proportion of the hydroxyl groups of the glycerols have reacted with a mixture of fatty acids such as stearic acid, capric acid, isostearic acid and 12-hydroxystearic acid, such as those notably marketed under the brand name Softisan 649 by the company Sasol;
esters of phytosterol;
esters of pentaerythritol;
esters fowled from:
   at least one C₁₆₋₄₀ alcohol, at least one of the alcohols being a Guerbet alcohol and
   a diacid dimer formed from at least one unsaturated C₁₈₋₄₀ fatty acid,

as the ester of dimer of tallol fatty acids comprising 36 carbon atoms and of a mixture i) of Guerbet alcohols comprising 32 carbon atoms and ii) of behenyl alcohol; the dimer ester of linoleic acid and of a mixture of two Guerbet alcohols, 2-tetradecyl-octadecanol (32 carbon atoms) and 2-hexadecyl-eicosanol (36 carbon atoms);
the non-crosslinked polyesters resulting from polycondensation between a dicarboxylic acid or a linear or branched C₄-C₅₀ polycarboxylic acid, and a diol or a C₂-C₅₀ polyol;
the polyesters resulting from esterification between a polycarboxylic acid and an aliphatic hydroxylated carboxylate such as Risocast DA-L and Risocast DA-H marketed by the Japanese company KOKYU ALCOHOL KOGYO, which are esters resulting from the reaction of esterification of hydrogenated castor oil with dilinoleic acid or isostearic acid; and
the aliphatic esters of ester resulting from esterification between an aliphatic hydroxylated carboxylate and an aliphatic carboxylic acid, for example that sold under the trade name Salacos HCJS (V)-L by the company Nishing Oil.

A Guerbet alcohol is the reaction product from the Guerbet reaction, which is well known by a person skilled in the art. It is a reaction that converts a primary aliphatic alcohol to its β-alkylated dimeric alcohol with loss of one equivalent of water.

The aliphatic carboxylic acids described above generally comprise from 4 to 30 and preferably from 8 to 30 carbon atoms. They are preferably selected from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid, docosanoic acid, and mixtures thereof.

The aliphatic carboxylic acids are preferably branched.

The esters of hydroxylated aliphatic carboxylic acid are advantageously obtained from a hydroxylated aliphatic carboxylic acid having from 0.2 to 40 carbon atoms, preferably from 10 to 34 carbon atoms and more preferably from 12 to 28 carbon atoms, and from 1 to 20 hydroxyl groups; preferably from 1 to 10 hydroxyl groups and more preferably from 1 to 6 hydroxyl groups. The esters of hydroxylated aliphatic carboxylic acid are notably selected from:

a) the partial or total esters of linear, saturated monohydroxylated aliphatic monocarboxylic acids;

b) the partial or total esters of unsaturated monohydroxylated aliphatic monocarboxylic acids;

c) the partial or total esters of saturated monohydroxylated aliphatic polycarboxylic acids;

d) the partial or total esters of saturated polyhydroxylated aliphatic polycarboxylic acids;

e) the partial or total esters of C₂ to C₁₆ aliphatic polyols that have reacted with a mono- or polyhydroxylated aliphatic mono- or polycarboxylic acid,

f) and mixtures thereof.

The aliphatic esters of ester are advantageously selected from:
the ester resulting from the reaction of esterification of hydrogenated castor oil with isostearic acid in proportions 1 to 1 (1/1), which is called monoisostearate of hydrogenated castor oil,
the ester resulting from the reaction of esterification of hydrogenated castor oil with isostearic acid in proportions 1, to 2 (½), which is called diisostearate of hydrogenated castor oil,
the ester resulting from the reaction of esterification of hydrogenated castor oil with isostearic acid in proportions 1 to 3 (⅓), which is called triisostearate of hydrogenated castor oil,
and mixtures thereof.

Preferably, the pasty compound is selected from compounds of vegetable origin.

Among the latter, we may notably mention isomerized jojoba oil such as the partially hydrogenated isomerized trans jojoba oil manufactured or marketed by the company Desert Whale under the commercial reference Iso-Jojoba-50®, orange wax such as, for example, that marketed under the reference Orange Peel Wax by the company Koster Keunen, cupuacu butter (Rain forest RF3410), murumuru butter from the company Beraca Sabara), karite butter, partially hydrogenated olive oil such as, for example, the compound marketed under the reference Beurrolive by the company Soliance, cocoa butter, mango oil such as, for example, Lipex 302 from the company Aarhuskarlshamn.

According to a particular embodiment, a composition according to the invention comprises karite butter.

The pasty compound or compounds can be present in a larger amount in the range from 0.1 to 20 wt. %, notably from 0.5 to 10 wt. %, relative to the total weight of the composition. The fatty phase of a composition according to the invention can if necessary optionally comprise at least one or more oils or organic solvents.

Oils or Organic Solvents

“Oil or organic solvent” means, in the sense of the application, a non-aqueous substance that is liquid at room temperature (25° C.) and atmospheric pressure (760 mmHg).

The oil can be selected from the volatile oils and/or the non-volatile oils, and mixtures thereof.

The oil or oils can be present at a content in the range from 1 to 50 wt. %, preferably from 5 to 30 wt. % relative to the total weight of the composition.

“Volatile oil” means, in the sense of the invention, an oil that can evaporate in contact with keratin fibres in less than one hour, at room temperature and atmospheric pressure. The volatile organic solvent or solvents and the volatile oils of the invention are organic solvents and volatile cosmetic oils, liquid at room temperature, having a non-zero vapour pressure, at room temperature and atmospheric pressure, in particular in the range from 0.13 Pa to 40 000 Pa (10⁻³ to 300 mmHg), in particular in the range from 1.3 Pa to 13 000 Pa (0.01 to 100 mmHg), and more particularly in the range from 1.3 Pa to 1300 Pa (0.01 to 10 mmHg).

“Non-volatile oil” means an oil that remains on keratin fibres at room temperature and atmospheric pressure for at least several hours and notably has a vapour pressure below 10⁻³ mmHg (0.13 Pa).

These oils can be hydrocarbon oils, silicone oils, fluorinated, oils, or mixtures thereof.

Volatile Oil

The composition according to the invention can comprise at least one volatile oil. This volatile oil can be a hydrocarbon oil. The volatile hydrocarbon oil can be selected from hydrocarbon oils having from 7 to 16 carbon atoms. The volatile hydrocarbon oil can be present in the composition according to the invention at a content in the range from 0.1 to 90 wt. %, relative to the total weight of the composition, preferably in the range from 1 to 70 wt. %, and preferably in the range from 5 to 70 wt. %, or even from 5 to 50 wt. %.

The composition according to the invention can contain one or more volatile branched alkane(s). “One or more volatile branched alkane(s)” means indiscriminately “one or more volatile branched alkane oils”.

As volatile hydrocarbon oil having from 7 to 16 carbon atoms, we may notably mention branched C₈-C₁₆ alkanes such as C₈-C₁₆ iso-alkanes (also called isoparaffins), isododecane, isodecane, isohexadecane and for example the oils sold under the trade names Isopars or Permethyls, the branched C₈-C₁₆ esters such as iso-hexyl neopentanoate, and mixtures thereof. Preferably, the volatile hydrocarbon oil having from 8 to 16 carbon atoms is selected from isododecane, isodecane, isohexadecane and mixtures thereof, and is notably isododecane.

The composition according to the invention can contain one or more volatile linear alkane(s). “One or more volatile linear alkane(s)” means indiscriminately “one or more volatile linear alkane oil(s)”.

A volatile linear alkane suitable for the invention is liquid at room temperature (about 25° C.) and at atmospheric pressure (760 mmHg).

“Volatile linear alkane” suitable for the invention means a cosmetic linear alkane, which can evaporate in contact with skin in less than one hour, at room temperature (25° C.) and atmospheric pressure (760 mmHg, i.e. 101 325 Pa), which is liquid at room temperature, notably having a rate of evaporation in the range from 0.01 to 15 mg/cm²/min, at room temperature (25° C.) and atmospheric pressure (760 mmHg).

Preferably, the “volatile linear alkanes” suitable for the invention have a rate of evaporation in the range from 0.01 to 3.5 mg/cm²/min, at room temperature (25° C.) and atmospheric pressure (760 mmHg).

Preferably, the “volatile linear alkanes” suitable for the invention have a rate of evaporation in the range from 0.01 to 1.5 mg/cm²/min, at room temperature (25° C.) and atmospheric pressure (760 mmHg).

More preferably, the “volatile linear alkanes” suitable for the invention have a rate of evaporation in the range from 0.01 to 0.8 mg/cm²/min, at room temperature (25° C.) and atmospheric pressure (760 mmHg).

Even more preferably, the “volatile linear alkanes” suitable for the invention have a rate of evaporation in the range from 0.01 to 0.3 mg/cm²/min, at room temperature (25° C.) and atmospheric pressure (760 mmHg).

Even more preferably, the “volatile linear alkanes” suitable for the invention have a rate of evaporation in the range from 0.01 to 0.12 mg/cm²/min; at room temperature (25° C.) and atmospheric pressure (760 mmHg).

The rate of evaporation of a volatile alkane according to the invention (and more generally of a volatile solvent) can notably be evaluated by means of the protocol described in WO 06/013413, and more particularly by means of the protocol described below.

Put 15 g of volatile hydrocarbon solvent in a crystallizing dish (diameter: 7 cm) placed on a balance that is in a chamber of about 0.3 m³ with controlled temperature (25° C.) and humidity (relative humidity 50%).

Allow the liquid to evaporate freely, without stirring, providing ventilation by a fan (PAPST-MOTOREN, reference 8550 N, speed 2700 rev/min) arranged in a vertical position above the crystallizing dish containing the volatile hydrocarbon solvent, with the blades directed towards the crystallizing dish, at a distance of 20 cm relative to the bottom of the crystallizing dish.

Measure the mass of the volatile hydrocarbon solvent remaining in the crystallizing dish at regular time intervals.

The evaporation profile of the solvent is then obtained by plotting the curve of the amount of product evaporated (in mg/cm²) as a function of time (in min.).

The rate of evaporation, which corresponds to the tangent at the origin of the curve obtained, is then calculated. The rates of evaporation are expressed in mg of volatile solvent evaporated per unit area (cm²) and per unit time (minute).

According to a preferred embodiment, the “volatile linear alkanes” suitable for the invention have a vapour pressure (also called saturated vapour pressure) that is non-zero, at room temperature, in particular a vapour pressure in the range from 0.3 Pa to 6000 Pa.

Preferably, the “volatile linear alkanes” suitable for the invention have a vapour pressure in the range from 0.3 to 2000 Pa, at room temperature (25° C.).

Preferably, the “volatile linear alkanes” suitable for the invention have a vapour pressure in the range from 0.3 to 1000 Pa, at room temperature (25° C.).

More preferably, the “volatile linear alkanes” suitable for the invention have a vapour pressure in the range from 0.4 to 600 Pa, at room temperature (25° C.).

Preferably, the “volatile linear alkanes” suitable for the invention have a vapour pressure in the range from 1 to 200 Pa, at room temperature (25° C.).

Even more preferably, the “volatile linear alkanes” suitable for the invention have a vapour pressure in the range from 3 to 60 Pa, at room temperature (25° C.).

According to one embodiment, a volatile linear alkane suitable for the invention can have a flash point in the range from 30 to 120° C., and more particularly from 40 to 100° C. The flash point is in particular measured according to standard ISO 3679.

According to one embodiment, an alkane suitable for the invention can be a volatile linear alkane comprising 7 to 14 carbon atoms.

Preferably, the “volatile linear alkanes” suitable for the invention have from 8 to 14 carbon atoms.

Preferably, the “volatile linear alkanes” suitable for the invention have from 9 to 14 carbon atoms.

Preferably, the “volatile linear alkanes” suitable for the invention have from 10 to 14 carbon atoms.

Preferably, the “volatile linear alkanes” suitable for the invention have from 11 to 14 carbon atoms.

According to an advantageous embodiment, the “volatile linear alkanes” suitable for the invention have a rate of evaporation, as defined above, in the range from 0.01 to 3.5 mg/cm²/min, at room temperature (25° C.) and atmospheric pressure (760 mmHg), and comprise from 8 to 14 carbon atoms.

A volatile linear alkane suitable for the invention can advantageously be of vegetable origin.

Preferably, volatile linear alkane or mixture of volatile linear alkanes present in the composition according to the invention comprises at least one isotope ¹⁴C of carbon (carbon 14), in particular the ¹⁴C isotope can be present in a ¹⁴C/¹²C ratio greater than or equal to 1×10⁻¹⁶, preferably greater than or equal to 1×10⁻¹⁵, more preferably greater than or equal to 7.5×10⁻¹⁴, and even more preferably greater than or equal to 1.5×10⁻¹³. Preferably, the ¹⁴C/¹²C ratio is from 6×10⁻¹³ to 1.2×10⁻¹².

The quantity of ¹⁴C isotopes in the volatile linear alkane or the mixture of volatile linear alkanes can be determined by methods known by a person skilled in the art such as the Libby counting method, liquid scintillation spectrometry or accelerator mass spectrometry.

Such an alkane can be obtained, directly or in several stages, from a vegetable raw material such as an oil, a butter, a wax, etc.

As examples of alkanes suitable for the invention, we may mention the alkanes described in the patent applications of the company Cognis WO 2007/068371, or WO2008/155059 (mixtures of different alkanes, differing by at least one carbon). These alkanes are obtained from fatty alcohols, themselves obtained from copra oil or palm oil.

As examples of linear alkanes suitable for the invention, we may mention n-heptane (C7), n-octane (C8), n-nonane (C9), n-decane (C10), n-undecane (C11), n-dodecane (C12), n-tridecane (C13), n-tetradecane (C14), and mixtures thereof. According to a particular embodiment, the volatile linear alkane is selected from n-nonane, n-undecane, n-dodecane, n-tridecane, n-tetradecane, and mixtures thereof.

According to a preferred embodiment, we may mention the mixtures of n-undecane (C11) and of n-tridecane (C13) obtained in examples 1 and 2 of application WO2008/155059 of the company Cognis.

We may also mention n-dodecane (C12) and n-tetradecane (C14) sold by Sasol respectively under the references PARAFOL 12-97 and PARAFOL 14-97, and mixtures thereof.

It will be possible to use the volatile linear alkane alone.

Alternatively or preferably, it will be possible to use a mixture of at least two different volatile linear alkanes, differing from one another by a number of carbons n of at least 1, in particular differing from one another by a number of carbons of 1 or 2.

According to a first embodiment, a mixture of at least two different volatile linear alkanes having from 10 to 14 carbon atoms and differing from one another by a number of carbons of at least 1 is used. As examples, we may notably mention the mixtures of volatile linear alkanes C₁₀/C₁₁, C₁₁/C₁₂, or C₁₂/C₁₃.

According to another embodiment, a mixture of at least two different volatile linear alkanes having from 10 to 14 carbon atoms and differing from one another by a number of carbons of at least 2 is used. As examples, we may notably mention the mixtures of volatile linear alkanes C₁₀/C₁₂, or C₁₂/C₁₄, for an even number of carbons n and the mixture C₁₁/C₁₃ for an odd number of carbons n.

According to a preferred embodiment, a mixture of at least two different volatile linear alkanes having from 10 to 14 carbon atoms and differing from one another by a number of carbons of at least 2, and in particular a mixture of volatile linear alkanes C₁₁/C₁₃ or a mixture of volatile linear alkanes C₁₂/C₁₄, is used

Other mixtures combining more than 2 volatile linear alkanes according to the invention, for example a mixture of at least 3 different volatile linear alkanes having from 7 to 14 carbon atoms and differing from one another by a number of carbons of at least 1, also form part of the invention, but mixtures of 2 volatile linear alkanes according to the invention are preferred (binary mixtures), said 2 volatile linear alkanes preferably representing more than 95% and more preferably more than 99 wt % of the total content of volatile linear alkanes in the mixture. According to a particular embodiment of the invention, in a mixture of volatile linear alkanes, the volatile linear alkane having the smallest number of carbons is predominant in the mixture.

According to another embodiment of the invention, a mixture of volatile linear alkanes is used in which the volatile linear alkane having the largest number of carbons is predominant in the mixture.

As examples of mixtures suitable for the invention, we may notably mention the following mixtures:
from 50 to 90 wt. %, preferably from 55 to 80 wt. %, more preferably from 60 to 75 wt. % of Cn volatile linear alkane with n in the range from 7 to 14,
from 10 to 50 wt. %, preferably from 20 to 45 wt. %, preferably from 24 to 40 wt. %, of Cn+x volatile linear alkane with x greater than or equal to 1, preferably x=1 or x=2, with n+x between 8 and 14, relative to the total weight of alkanes in said mixture.

In particular, said mixture of alkanes according to the invention contains:
less than 2 wt. %, preferably less than 1 wt. % of branched hydrocarbons,
and/or less than 2 wt. %, preferably less than 1 wt. % of aromatic hydrocarbons,
and/or less than 2 wt. %, preferably less than 1 wt. % and preferably less than 0.1 wt. % of unsaturated hydrocarbons in the mixture.

More particularly, a volatile linear alkane suitable for the invention can be used in the form of an n-undecane/n-tridecane mixture.

In particular, a mixture of volatile linear alkanes will be used comprising:
from 55 to 80 wt. %, preferably from 60 to 75 wt. % of C₁₁ volatile linear alkane (n-undecane)
from 20 to 45 wt. %, preferably from 24 to 40 wt. % of C₁₃ volatile linear alkane (n-tridecane)

relative to the total weight of the alkanes in said mixture.

According to a particular embodiment, the mixture of alkanes is an n-undecane/n-tridecane mixture. In particular, such a mixture can be obtained according to example 1 or example 2 of WO 2008/155059.

According to another particular embodiment, the n-dodecane sold under the reference PARAFOL 12-97 by SASOL is used.

According to another particular embodiment, the n-tetradecane sold under the reference PARAFOL 14-97 by SASOL is used.

According to yet another embodiment, a mixture of n-dodecane and n-tetradecane is used.

As a variant or additionally, the composition produced can comprise at least one volatile silicone oil or solvent, compatible with cosmetic use.

“Silicone oil” means an oil containing at least one silicon atom, and notably containing Si—O groups. According to one embodiment, said composition comprises less than 10 wt. % of non-volatile silicone oil(s), relative to the total weight of the composition, preferably less than 5 wt. %, or even is free from silicone oil.

As volatile silicone oil, we may mention cyclic polysiloxanes, linear polysiloxanes and mixtures thereof. As volatile linear polysiloxanes, we may mention hexamethyldisiloxane, octamethyltrisiloxane, decamethyltetrasiloxane, tetradecamethylhexasiloxane and hexadecamethylheptasiloxane. As cyclic volatile polysiloxanes, we may mention hexamethylcyclotrisiloxane, octamethylcyclotetrasiloxane, decamethylcyclopentasiloxane and dodecamethylcyclohexasiloxane.

As a variant or additionally, the composition produced can comprise at least one volatile fluorinated oil.

Fluorinated oil means an oil containing at least one fluorine atom.

As volatile fluorinated oil, we may mention nonafluoromethoxybutane or perfluoromethylcyclopentane, and mixtures thereof.

Non-Volatile Oils

The non-volatile oils can notably be selected from non-volatile hydrocarbon oils, fluorinated oils and/or silicone oils.

As non-volatile hydrocarbon oil, we may notably mention:
hydrocarbon oils of animal origin,
hydrocarbon oils of vegetable origin, such as phytostearyl esters, such as phytostearyl oleate, phytostearyl isostearate and lauroyl/octyldodecyl/phytostearyl glutamate (AJINOMOTO, ELDEW PS203), triglycerides constituted of esters of fatty acids and of glycerol, in particular, in which the fatty acids can have chain lengths in the range from C₄ to C₃₆, and notably from C₁₈ to C₃₆, and said oils can be linear or branched, saturated or unsaturated; these oils can notably be heptanoic or octanoic triglycerides, karite oil, alfalfa oil, poppy oil, Chinese okra oil, millet oil, barley oil, quinoa oil, rye oil, candlenut oil, passionflower oil, karite butter oil, aloes oil, sweet almond oil, peach kernel oil, peanut oil, argan oil, avocado oil, baobab oil, borage oil, broccoli oil, calendula oil, camelina oil, carrot oil, safflower oil, hemp oil, colza oil, cotton oil, copra oil, cucurbit seed oil, wheatgerm oil, jojoba oil, lily oil, macadamia oil, maize oil, meadowfoam oil, St John's wort oil, scented coconut oil, hazlenut oil, apricot kernel oil, walnut oil, olive oil, evening primrose oil, palm oil, blackcurrant seed oil, kiwi seed oil, grapeseed oil, pistachio oil, Chinese okra oil, pumpkin oil, quinoa oil, musk rose oil, sesame oil, soya oil, sunflower oil, castor oil, and watermelon oil, and mixtures thereof, or triglycerides of caprylic/capric acids, such as those sold by the company STEARINERIES DUBOIS or those sold under the designations MIGLYOL 810®, 812® and 818® by the company DYNAMIT NOBEL,
synthetic ethers having from 10 to 40 carbon atoms;
synthetic esters, such as oils of formula R₁COOR₂, in which R₁ represents a residue of a linear or branched fatty acid having from 1 to 40 carbon atoms and R₂ represents a hydrocarbon chain, notably branched containing from 1 to 40 carbon atoms provided that R₁+R₂ is ≧10. The esters can notably be selected from the esters of an alcohol and a fatty acid, for example:
   cetostearyl octanoate, the esters of isopropyl alcohol, such as isopropyl myristate, isopropyl palmitate, ethyl palmitate, 2-ethylhexyl palmitate, isopropyl stearate or isostearate, isostearyl isostearate, actyl stearate, hydroxylated esters, such as isostearyl lactate; octyl hydroxystearate, diisopropyl adipate, heptanoates, and notably isostearyl heptanoate, octanoates, decanoates or ricinoleates of alcohols or of polyalcohols, such as propylene glycol dioctanoate, cetyl octanoate, tridecyl octanoate, ethyl-2-hexyl-4-diheptanoate and palmitate, alkyl benzoate, polyethylene glycol diheptanoate, propylene glycol diethyl-2-dihexanoate and mixtures thereof, benzoates of C₁₂-C₁₅ alcohols, hexyl laurate, esters of neopentanoic acid, such as isodecyl neopentanoate, isotridecyl neopentanoate, isostearyl neopentanoate, octyldocecyl neopentanoate, esters of isononanoic acid, such as isononyl isononanoate, isotridecyl isononanoate, octyl isononanoate, hydroxylated esters such as isostearyl lactate, diisostearyl malate;
esters of polyols and esters of pentaerythritol, such as dipentaerythritol tetrahydroxystearate/tetraisostearate,
esters of diol dimers and of diacid dimers, such as Lusplan DD-DA5® and Lusplan DD-DA7®, marketed by the company NIPPON FINE CHEMICAL and described in application US 2004-175338,
copolymers of dial dimer and of diacid dimer and their esters, such as copolymers of dilinoleyl dial dimers/dilinoleic dimers and their esters, for example Plandool-G,
copolymers of polyols and of diacid dimers, and their esters, such as Hailuscent ISDA,
fatty alcohols that are liquid at room temperature with a branched and/or unsaturated carbon chain having from 12 to 26 carbon atoms, such as 2-octyldodecanol, isostearyl alcohol, oleic alcohol, 2-hexyldecanol, 2-butyloctanol, and 2-undecylpentadecanol,
C₁₂-C₂₂ higher fatty acids, such as oleic acid, linoleic acid, linolenic acid and mixtures thereof, and
dialkyl carbonates, the 2 alkyl chains being identical or, different, such as dicaprylyl carbonate marketed under the designation CETIOL CC®, by COGNIS,
oils of high molar mass having, in particular, a molar mass in the range from about 400 to about 10 000 g/mol, in particular from about 650 to about 10 000 g/mol, in particular from about 750 to about 7500 g/mol, and more particularly in the range from about 1000 to about 5000 g/mol. As oil of high molar mass usable in the present invention, we may notably mention the oils selected from:
   lipophilic polymers,
   esters of linear fatty acids having a total number of carbons in the range from 35 to 70,
   hydroxylated esters,
   aromatic esters,
   esters of fatty alcohols or of branched C₂₄-C₂₈ fatty acids,
   silicone oils,
   oils of vegetable origin,
   and mixtures thereof.

For example, an oil of high molar mass can be selected from:
a) lipophilic polymers, such as:
   polybutylenes, such as INDOPOL H-100 (of molar mass MM=965 g/mol), INDOPOL H-300 (MM=1340 g/mol), INDOPOL H-1500 (MM=2160 g/mol) marketed or manufactured by the company AMOCO,
   polyisobutylenes, for example hydrogenated, such as PANALANE H-300 E marketed or manufactured by the company AMOCO (MM=1340 g/mol), VISEAL 20000 marketed or manufactured by the company SYNTEAL (MM=6000 g/mol), REWOPAL PIB 1000 marketed or manufactured by the company WITCO (MM=1000 g/mol),
   polydecenes and hydrogenated polydecenes, such as: PURESYN 10 (MM=723 g/mol), PURESYN 150 (MM=9200 g/mol) marketed or manufactured by the company MOBIL CHEMICALS,
   vinylpyrrolidone copolymers, such as: the vinylpyrrolidone/1-hexadecene copolymer ANTARON V-216 marketed or manufactured by the company ISP (MM=7300 g/mol), and copolymers of polyvinylpyrrolidone (PVP), such as the copolymers of a C₂-C₃₀, such as C₃-C₂₂, alkene and combinations thereof, can be used. As examples of PVP copolymers that can be used in the invention, we may mention the copolymers: PVP/vinyl laurate, PVP/vinyl stearate, butylated PVP, PVP/hexadecene, PVP/triacontene or PVP/acrylic acid/lauryl methacrylate,
b) esters, such as:
   esters of linear fatty acids having a total number of carbons in the range from 35 to 70, such as pentaerythrityl tetrapelargonate (MM=697 g/mol), hydroxylated esters, such as polyglycerol-2 triisostearate (MM=965 g/mol),
   aromatic esters, such as tridecyl trimellitate (MM=757 g/mol),
   esters of fatty alcohols or of branched C₂₄-C₂₈ fatty acids, such as those described in U.S. Pat. No. 6,491,927 and the esters of pentaerythritol, and notably triisoarachidyl citrate (MM=1033.76 g/mol), pentaerythrityl tetraisononanoate (MM=697 g/mol), glyceryl triisostearate (MM=891 g/mol), glyceryl tridecyl-2-tetradecanoate (MM=1143 g/mol), pentaerythrityl tetraisostearate (MM=1202 g/mol), polyglyceryl-2-tetraisostearate (MM=1232 g/mol) or pentaerythrityl tetradecyl-2-tetradecanoate (MM=1538 g/mol),
   dimer diol esters and polyesters, such as the esters of dimer diol and of fatty acid, and the esters of dimer dials and of diacid, such as Lusplan DD-DA5® and Lusplan DD-DA7® marketed by the company NIPPON FINE CHEMICAL and described in application US 2004-175338,
c) silicone oils, such as the phenylated silicones such as BELSIL PDM 1000 from the company WACKER (MM=9000 g/mol). Other non-volatile silicone oils usable in the composition according to the invention can be non-volatile polydimethylsiloxanes (PDMS), PDMS having pendant and/or silicone-chain-end alkyl or alkoxy groups, groups each having from 2 to 24 carbon atoms, phenylated silicones, such as phenyl trimethicones, phenyl dimethicones, phenyl trimethylsiloxy diphenylsiloxanes, diphenyl dimethicones, diphenyl methyldiphenyl trisiloxanes, and 2-phenylethyl trimethylsiloxysilicates, dimethicones or phenyltrimethicone with viscosity less than or equal to 100 cSt, and mixtures thereof,

as well as mixtures of oils a) and/or b) and/or c).

The fluorinated oils usable in the invention are notably fluorosilicone oils, fluorinated polyethers, fluorinated silicones as described in document EP-A-847752.

The compositions according to the invention can further comprise any ingredient used conventionally in the fields in question and more particularly in the field of mascaras and/or nail varnishes, for example pigments or nacres, film-forming polymers, gelling agents, fillers and/or of fibres.

Pigments

“Pigments” are to be understood as white or coloured particles, mineral or organic, insoluble in an aqueous medium, intended to colour and/or opacify the composition and/or the resultant film.

Pigments can be white or coloured, mineral and/or organic.

The pigment can be an organic pigment. “Organic pigment” means any pigment that corresponds to the definition in the chapter on organic pigments in Ullmann's encyclopaedia. The organic pigment can notably be selected from the nitroso, nitro, azo, xanthene, quinoline, anthraquinone, phthalocyanine, of the metallic complex type, isoindolinone, isoindoline, quinacridone, perinone, perylene, diketopyrrolopyrrole, thioindigo, dioxazine, triphenylmethane, quinophthalone compounds.

The organic pigment or pigments can be selected for example from carmine, carbon black, aniline black, melanin, azo yellow, quinacridone, phthalocyanine blue, red sorghum, blue pigments codified in the Color Index under the references CI-42090, 69800, 69825, 73000, 74100, 74160, yellow pigments codified in the Color Index under the references CI 11680, 11710, 15985, 19140, 20040, 21100, 21108, 47000, 47005, green pigments codified in the Color Index under the references CI-61565, 61570, 74260, orange pigments codified in the Color Index under the references CI 11725, 15510, 45370, 71105, red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, pigments obtained by oxidative polymerization of indole and phenol derivatives, as described in patent FR 2 679 771.

These pigments can also be in the form of composite pigments as described in patent EP 1 184 426. These composite pigments can be composed notably of particles having an inorganic core covered at least partially with an organic pigment and at least one binder ensuring fixation of the organic pigments on the core.

The pigment can also be a lake. “Lake” means dyes that have been rendered insoluble, adsorbed on insoluble particles, the whole thus obtained remaining insoluble during use.

The inorganic substrates on which the dyes are adsorbed are for example alumina, silica, calcium and sodium borosilicate or calcium and aluminium borosilicate, and aluminium.

Among the organic dyes, we may mention carmine. We may also mention the products known by the following designations: D & C Red 21 (CI 45 380), D & C Orange 5 (CI 45 370), D & C Red 27 (CI 45 410), D & C Orange 10 (CI 45 425), D & C Red 3 (CI-430), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 1 O (CI 77 002), D & C Green 3 (CI 42 053), D & C Blue 1 (CI 42 090).

As examples of lakes, we may mention the product known by the following designation: D & C Red 7 (CI 15 850:1).

The pigment can be a mineral pigment. Mineral pigment means any pigment that corresponds to the definition in the chapter on inorganic pigments in Ullmann's encyclopaedia. We may mention, among mineral pigments for use in the present invention, oxides of zirconium or of cerium, as well as oxides of zinc, of iron (black, yellow or red) or of chromium, manganese violet, ultramarine blue, chromium hydroxide and ferric blue, titanium dioxide, metal powders such as aluminium powder and copper powder. The following mineral pigments can also be used: Ta₂O₅, Ti₃O₅, Ti₂O₃, TiO, ZrO₂ mixed with TiO₂, ZrO₂, Nb₂O₅, CeO₂, ZnS.

The particle size of the pigment for use within the scope of the present invention is generally between 10 nm and 10 μm, preferably between 20 nm and 5 μm, and more preferably between 30 nm and 1 μm.

Film-Forming Polymers

Among the film-forming polymers usable in the compositions of the present invention, we may mention synthetic polymers, of the radical type or of the polycondensate type, polymers of natural origin, and mixtures thereof.

Radical film-forming polymer means a polymer obtained by polymerization of monomers with an unsaturation, notably ethylenic, each monomer being capable of homopolymerization (in contrast to the polycondensates).

The film-forming polymers of the radical type can notably be vinylic polymers, or copolymers, notably acrylic polymers.

The vinylic film-forming polymers can result from the polymerization of monomers with an ethylenic unsaturation having at least one acid group and/or esters of these acid monomers and/or amides of these acid monomers.

As monomer bearing an acid group, α,β-ethylenic unsaturated carboxylic acids can be used, such as acrylic acid, methacrylic acid, crotonic acid, maleic acid, itaconic acid. It is preferable to use (meth)acrylic acid and crotonic acid, and more preferably (meth)acrylic acid.

The esters of acid monomers are advantageously selected from the esters of (meth)acrylic acid (also called (meth)acrylates), notably alkyl(meth)acrylates, in particular of C₁-C₃₀, preferably C₁-C₂₀ alkyl; aryl(meth)acrylates, in particular of C₆-C₁₀ aryl, hydroxyalkyl(meth)acrylates, in particular of C₂-C₆ hydroxyalkyl.

Among the alkyl(meth)acrylates, we may mention methyl methacrylate, ethyl methacrylate, butyl methacrylate, isobutyl methacrylate, ethyl-2-hexyl methacrylate, lauryl methacrylate, cyclohexyl methacrylate.

Among the hydroxyalkyl(meth)acrylates, we may mention hydroxyethyl acrylate, 2-hydroxypropyl acrylate, hydroxyethyl methacrylate, 2-hydroxypropyl methacrylate.

Among the aryl(meth)acrylates, we may mention benzyl acrylate and phenyl acrylate.

The esters of (meth)acrylic acid that are particularly preferred are the alkyl (meth)acrylates.

According to the present invention, the alkyl group of the esters can be either fluorinated, or perfluorinated, i.e. some or all of the hydrogen atoms of the alkyl group are substituted with fluorine atoms.

As amides of acid monomers, we may mention for example (meth)acrylamides, and notably N-alkyl(meth)acrylamides, in particular of C₂-C₁₂ alkyl. Among N-alkyl (meth)acrylamides, we may mention N-ethyl acrylamide, N-t-butyl acrylamide, N-t-octyl acrylamide and N-undecylacrylamide.

The vinylic film-forming polymers can also result from the homopolymerization or copolymerization of monomers selected from the vinyl esters and styrene monomers. In particular, these monomers can be polymerized with acid monomers and/or their esters and/or their amides, such as those mentioned previously.

As examples of vinyl esters, we may mention vinyl acetate, vinyl neodecanoate, vinyl pivalate, vinyl benzoate and vinyl t-butyl benzoate.

As styrene monomers, we may mention styrene and alpha-methylstyrene.

Among film-forming polycondensates, we may mention polyurethanes, polyesters, polyester amides, polyamides, and epoxy ester resins, polyureas.

The polyurethanes can be selected from anionic, cationic, non-ionic or amphoteric polyurethanes, polyurethane-acrylics, polyurethane-polyvinylpyrrolidones, polyester-polyurethanes, polyether-polyurethanes, polyureas, polyurea-polyurethanes, and mixtures thereof.

The polyesters can be obtained, in a known manner, by polycondensation of dicarboxylic acids with polyols, notably dials.

The dicarboxylic acid can be aliphatic, alicyclic or aromatic. We may mention as examples of such acids: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornane dicarboxylic acid, diglycolic acid, thiodipropionic acid, 2,5-naphthalenedicarboxylic acid, 2,6-naphthalenedicarboxylic acid. These dicarboxylic acid monomers can be used alone or in combination with at least two dicarboxylic acid monomers. Among these monomers, preferably phthalic acid, isophthalic acid, and terephthalic acid are selected.

The diol can be selected from the aliphatic, alicyclic, aromatic diols. It is preferable to use a diol selected from: ethylene glycol, diethylene glycol, triethylene glycol, 1,3-propanediol, cyclohexane dimethanol, 4-butanediol. As other polyols, it is possible to use glycerol, pentaerythritol, sorbitol, trimethylol propane.

The polyester amides can be obtained similarly to the polyesters, by polycondensation of diacids with diamines or amino alcohols. As diamine, it is possible to use ethylenediamine, hexamethylenediamine, meta- or para-phenylenediamine. As aminoalcohol, it is possible to use monoethanolamine.

The polyester can further comprise at least one monomer bearing at least one group —SO₃M, with M representing a hydrogen atom, an ammonium ion NH₄⁺ or a metal ion, for example an ion Na⁺, Li⁺, K⁺, Mg²⁺, Ca²⁺, Cu²⁺, Fe²⁺, Fe³⁺. Notably a bifunctional aromatic monomer bearing said group —SO₃M can be used.

The aromatic nucleus of the bifunctional aromatic monomer additionally bearing a group —SO₃M as described above can be selected for example from the benzene, naphthalene, anthracene, diphenyl, oxydiphenyl, sulphonyldiphenyl, methylenediphenyl rings. We may Mention as examples of bifunctional aromatic monomer additionally bearing a group —SO₃M: sulphoisophthalic acid, sulphoterephthalic acid, sulphophthalic acid, 4-sulphonaphthalene-2,7-dicarboxylic acid.

It is preferable to use copolymers based on isophthalate/sulphoisophthalate, and more particularly copolymers obtained by condensation of diethylene glycol, cyclohexane dimethanol, isophthalic acid, sulphoisophthalic acid.

The polymers of natural origin, optionally modified, can be selected from shellac resin, sandarac gum, dammars, elemis, copals, cellulosic polymers, and mixtures thereof.

According to a first embodiment of the invention, the film-forming polymer can be a water-soluble polymer and can then be present in the aqueous continuous phase of an emulsion according to the invention.

According to another variant, the film-forming polymer can be a polymer solubilized in a liquid fatty phase comprising oils or organic solvents such as those described hereunder (it is then said that the film-forming polymer is a fat-soluble polymer). Preferably, the liquid fatty phase comprises a volatile oil, optionally mixed with a non-volatile oil, and the oils can be selected from the oils mentioned below.

As examples of fat-soluble polymer, we may mention the copolymers of vinyl ester (the vinylic group being joined directly to the oxygen atom of the ester group and the vinyl ester having a saturated, linear or branched hydrocarbon radical, with from 1 to 19 carbon atoms, bound to the carbonyl of the ester group) and of at least one other monomer, which can be a vinyl ester (different from the vinyl ester already present), an α-olefin (having from 8 to 28 carbon atoms), an alkylvinyl ether (whose alkyl group has from 2 to 18 carbon atoms), or an allylic or methallylic ester (having a saturated, linear or branched hydrocarbon radical, with from 1 to 19 carbon atoms, bound to the carbonyl of the ester group).

These copolymers can be crosslinked by crosslinking agents which can be either of the vinylic type, or of the allylic or methallylic type, such as tetraallyloxyethane, divinylbenzene, divinyl octanedioate, divinyl dodecanedioate, and divinyl octadecanedioate.

As examples of these copolymers, we may mention the copolymers: vinyl acetate/allyl stearate, vinyl acetate/vinyl laurate, vinyl acetate/vinyl stearate, vinyl acetate/octadecene, vinyl acetate/octadecylvinyl ether, vinyl propionate/allyl laurate, vinyl propionate/vinyl laurate, vinyl stearate/octadecene-1, vinyl acetate/dodecene-1, vinyl stearate/ethylvinyl ether, vinyl propionate/cetyl vinyl ether, vinyl stearate/allyl acetate, vinyl dimethyl-2,2-octanoate/vinyl laurate, allyl dimethyl-2,2-pentanoate/vinyl laurate, vinyl dimethyl propionate/vinyl stearate, allyl dimethyl propionate/vinyl stearate, vinyl propionate/vinyl stearate, crosslinked with 0.2% of divinyl benzene, vinyl dimethyl propionate/vinyl laurate, crosslinked with 0.2% of divinyl benzene, vinyl acetate/vinyl octadecyl ether, crosslinked with 0.2% of tetraallyloxyethane, vinyl acetate/allyl stearate, crosslinked with 0.2% of divinyl benzene, vinyl acetate/octadecene-1 crosslinked with 0.2% of divinyl benzene and allyl propionate/allyl stearate crosslinked with 0.2% of divinyl benzene.

As fat-soluble film-forming polymers, we may also mention the fat-soluble copolymers, and in particular those resulting from copolymerization of vinyl esters having from 9 to 22 carbon atoms or alkyl acrylates or methacrylates, the alkyl radicals having from 10 to 20 carbon atoms.

These fat-soluble copolymers can be selected from the copolymers of vinyl polystearate, of vinyl polystearate crosslinked by divinylbenzene, diallyl ether or diallyl phthalate, copolymers of stearyl poly(meth)acrylate, of polyvinyl laurate, of lauryl poly(meth)acrylate, and said poly(meth)acrylates can be crosslinked by dimethacrylate of ethylene glycol or of tetraethylene glycol.

The fat-soluble copolymers defined above are known and notably described in application FR-A-2232303; they can have a weight-average molecular weight in the range from 2000 to 500 000 and preferably from 4000 to 200 000.

We may also mention the fat-soluble homopolymers, and in particular those resulting from the homopolymerization of vinyl esters having from 9 to 22 carbon atoms or of alkyl acrylates or methacrylates, the alkyl radicals having from 2 to 24 carbon atoms.

As examples of fat-soluble homopolymers, we may notably mention: polyvinyl laurate and lauryl poly(meth)acrylates, and said poly(meth)acrylates can be crosslinked by dimethacrylate of ethylene glycol or of tetraethylene glycol.

According to an advantageous embodiment, a composition according to the invention comprises at least one polyvinyl laurate film-forming polymer.

As fat-soluble film-forming polymers usable in the invention, we may also mention the polyalkylenes and notably the copolymers of C₂-C₂₀ alkenes, such as polybutene, the alkylcelluloses with a linear or branched, saturated or unsaturated C₁ to C₈ alkyl radical such as ethylcellulose and propylcellulose, the copolymers of vinylpyrrolidone (VP) and notably the copolymers of vinylpyrrolidone and C₂ to C₄₀ and preferably C₃ to C₂₀ alkene. As examples of VP copolymer usable in the invention, we may mention the VP/vinyl acetate, VP/ethyl methacrylate, butylated polyvinylpyrolidone (PVP), VP/ethyl methacrylate/methacrylic acid, VP/eicosene, VP/hexadecene, VP/triacontene, VP/styrene, VP/acrylic acid/lauryl methacrylate copolymers.

We may also mention the silicone resins, generally soluble or swellable in silicone oils, which are crosslinked polymers of polyorganosiloxanes. The nomenclature of the silicone resins is known under the name “MDTQ”, the resin being described according to the different siloxane monomeric units that it comprises, each of the letters “MDTQ” characterizing a type of unit.

As examples of commercially available polymethylsilsesquioxane resins, we may mention those that are marketed by the company Wacker under the reference Resin MK such as Belsil PMS MK, and by the company SHIN-ETSU under the references KR-220L.

As siloxysilicate resins, we may mention the trimethylsiloxysilicate (TMS) resins such as those marketed under the reference SR1000 by the company General Electric or under the reference TMS 803 by the company Wacker. We may also mention the trimethylsiloxysilicate resins marketed in a solvent such as cyclomethicone, sold under the designation “KF-7312J” by the company Shin-Etsu, “DC 749”, “DC 593” by the company Dow Corning.

We may also mention copolymers of silicone resins such as those mentioned above with polydimethylsiloxanes, such as the pressure-sensitive adhesive copolymers marketed by the company Dow Corning under the reference BIO-PSA and described in document U.S. Pat. No. 5,162,410 or the silicone copolymers resulting from the reaction of a silicone resin, such as those described above, and a diorganosiloxane such as described in document WO 2004/073626.

It is also possible to use silicone polyamides of the polyorganosilaxane type such as those described in documents U.S. Pat. No. 5,874,069, U.S. Pat. No. 5,919,441, U.S. Pat. No. 6,051,216 and U.S. Pat. No. 5,981,680.

These silicone polymers can belong to the following two families:
polyorganosiloxanes having at least two groups capable of establishing hydrogen interactions, these two groups being located in the chain of the polymer, and/or
polyorganosiloxanes having at least two groups capable of establishing hydrogen interactions, these two groups being located on grafts or branchings.

According to one embodiment of the invention, the film-forming polymer is a film-forming linear ethylenic block polymer, which preferably comprises at least one first block and at least one second block having different glass transition temperatures (Tg), said first and second blocks being joined together by an intermediate block comprising at least one constituent monomer of the first block and at least one constituent monomer of the second block.

Advantageously, the first and second blocks of the block polymer are incompatible with one another.

Such polymers are described for example in documents EP 1 411 069 or WO 04/028488.

The film-forming polymer can also be present in a composition of the invention in the form of particles dispersed in an aqueous phase or in a non-aqueous solvent phase, generally known as a latex or pseudolatex. The techniques for preparing these dispersions are well known by a person skilled in the art.

As aqueous dispersion of film-forming polymer, it is possible to use the acrylic dispersions sold under the designations Neocryl XK-90®, Neocryl A-1070®, Neocryl A-1090®, Neocryl BT-62®, Neocryl A-1079® and Neocryl A-523® by the company AVECIA-NEORESINS, Dow Latex 432® by the company DOW CHEMICAL, Daitosol 5000 AD® or Daitosol 5000 SJ® by the company DAITO KASEI KOGYO; Syntran 5760® by the company Interpolymer, Allianz OPT by the company ROHM & HAAS, the aqueous dispersions of acrylic or styrene/acrylic polymers sold under the brand name JONCRYL® by the company JOHNSON POLYMER or the aqueous dispersions of polyurethane sold under the designations Neorez R-981° and Neorez R-974® by the company AVECIA-NEORESINS, Avalure UR-405®, Avalure UR-410®, Avalure TJR-425®, Avalure UR-450®, Sancure 875®, Sancure 861®, Sancure 878® and Sancure 2060® by the company GOODRICH, Impranil 85® by the company BAYER, Aquamere H-1511® by the company HYDROMER; the sulphopolyesters sold under the brand name Eastman AQ® by the company Eastman Chemical Products, the vinylic dispersions such as Mexomer PAM® from the company CHIMEX and mixtures thereof.

As examples of non-aqueous dispersions of film-forming polymer, we may mention the acrylic dispersions in isododecane such as Mexomer PAP® from the company CHIMEX, the dispersions of particles of a grafted ethylenic polymer, preferably acrylic, in a liquid fatty phase, the ethylenic polymer advantageously being dispersed in the absence of additional stabilizer on the surface of the particles as described notably in document WO 04/055081.

A composition according to the invention can also further comprise a plasticizer promoting the formation of a film with the film-forming polymer. Such a plasticizer can be selected from all the compounds known by a person skilled in the art as being capable of performing the required function.

Gelling Agents

A composition of the invention can also comprise at least one hydrophilic or water-soluble gelling agent.

As hydrophilic or water-soluble gelling agents, we may mention:
the homo- or copolymers of acrylic or methacrylic acids or their salts and their esters and in particular the products sold under the designations “VERSICOL F” or “VERSICOL K” by the company ALLIED COLLOID, “UTRAHOLD 8” by the company CIBA-GEIGY, the polyacrylic acids of the SYNTHALEN K type,
the copolymers of acrylic acid and acrylamide sold in the form of their sodium salt under the designations “RETEN” by the company HERCULES, the sodium polymethacrylate sold under the designation “DARVAN N^{o}7” by the company VANDERBILT, the sodium salts of polyhydroxycarboxylic acids sold under the designation “HYDAGEN F” by the company HENKEL,
the polyacrylic acids/alkyl acrylates copolymers of the PEMULEN type,
AMPS® (polyacrylamidomethyl propane sulphonic acid partially neutralized with ammonia and highly crosslinked) marketed by the company CLARIANT,
the AMPS®/acrylamide copolymers of the SEPIGEL or SIMULGEL type marketed by the company SEPPIC, and
the AMPS®/polyethoxylated alkyl methacrylates copolymers (crosslinked or not) and mixtures thereof.

As other examples of water-soluble polymeric gelling agents, we may mention:
proteins such as proteins of vegetable origin such as proteins from wheat, from soya; proteins of animal origin such as keratins, for example the keratin hydrolysates and the sulphonic keratins;
anionic, cationic, amphoteric or non-ionic polymers of chitin or of chitosan;
cellulose polymers such as hydroxyethylcellulose, hydroxypropylcellulose, methylcellulose, ethylhydroxyethylcellulose, carboxymethylcellulose, as well as the quaternized derivatives of cellulose;
vinyl polymers, such as polyvinylpyrrolidones, copolymers of methylvinylic ether and malic anhydride, copolymer of vinyl acetate and crotonic acid, copolymers of vinylpyrrolidone and vinyl acetate; copolymers of vinylpyrrolidone and caprolactam; polyvinyl alcohol;
associative polyurethanes such as the polymer C16-OE120-C16 from the company SERVO DELDEN (marketed under the name SER AD FX1100, molecule with urethane function and weight-average molecular weight of 1300), OE being an ethoxylated unit, Rheolate 205 with urea function sold by the company RHEOX or Rheolate 208 or 204 (these polymers being sold in the pure form) or DW 120613 from ROHM & HAAS with C₂₀ alkyl chain and with urethane bond, sold at 20% dry matter in water. It is also possible to use solutions or dispersions of these associative polyurethanes notably in water or in an aqueous-alcoholic medium. As examples of said polymers, we may mention SER AD fx1010, SER AD FX1035 and SER AD 1070 from the company SERVO DELDEN, Rheolate 255, Rheolate 278 and Rheolate 244 sold by the company RHEOX. It is also possible to use the product DW 1206F and DW 1206J, as well as Acrysol RM 184 or Acrysol 44 from the company ROHM & HAAS, or Borchigel LW 44 from the company BORCHERS,
polymers of natural origin, optionally modified, such as:
   gum arabic, guar gum, derivatives of xanthan, karaya gum;
   alginates and carrageenans;
   glycoaminoglycans, hyaluronic acid and its derivatives;
   shellac resin, sandarac gum, dammars, elemis, copals;
   deoxyribonucleic acid;
   mucopolysaccharides such as hyaluronic acid, chondroitin sulphates, and mixtures thereof.

Certain water-soluble film-forming polymers mentioned above can also perform the role of water-soluble gelling agent.

The hydrophilic gelling agents can be present in the compositions according to the invention at a content in the range from 0.05 to 40 wt. % relative to the total weight of the composition, preferably from 0.1 to 20% and more preferably from 0.5 to 15 wt. %.

Fillers

The composition according to the invention can also comprise at least one filler. These fillers notably serve for modifying the rheology or the texture of the composition.

The fillers can be mineral or organic of any shape, lamellar, spherical or oblong, regardless of the crystallographic form (for example leaf, cubic, hexagonal, orthorhombic, etc.). We may mention talc, mica, silica, silica surface-treated with a hydrophobic agent, kaolin, powders of polyamide (Nylon®) (Orgasol® from Atochem), of poly-3-alanine and of polyethylene, powders of tetrafluoroethylene polymers (Teflon®), lauroyl-lysine, starch, boron nitride, Hollow polymeric microspheres such as those of polyvinylidene chloride/acrylonitrile such as Expancel® (Nobel Industrie), of acrylic acid copolymers (Polytrap® from the company Dow Corning) and microbeads of silicone resin (Tospearls® from Toshiba, for example), particles of elastomeric polyorganosiloxanes, precipitated calcium carbonate, magnesium carbonate and magnesium hydrogen carbonate, hydroxyapatite, hollow silica microspheres (Silica Beads® from Maprecos), glass or ceramic microcapsules, metal soaps derived from organic carboxylic acids having from 8 to 22 carbon atoms, preferably from 12 to 18 carbon atoms, for example zinc, magnesium or lithium stearate, zinc laurate, magnesium myristate.

It is also possible to use a compound that can swell under the effect of heat and notably thermoexpandable particles such as unexpanded microspheres of vinylidene chloride/acrylonitrile/methyl methacrylate copolymer or of copolymer of acrylonitrile homopolymer, for example those marketed respectively under the references Expand® 820 DU 40 and Expancel®007WU by the company AKZO NOBEL.

The fillers can represent from 0.1 to 25 wt. %, in particular from 0.2 to 20 wt. % relative to the total weight of the composition.

Fibres

The compositions according to the invention can also comprise at least one fibre, notably making it possible, in the case of application of a composition of the invention in the form of mascara, to obtain an improvement of the lengthening effect.

“Fibre” is to be understood as an object of length L and diameter D such that L is greater than D, and preferably much greater than D, D being the diameter of the circle in which the fibre cross-section is inscribed. In particular, the ratio L/D (or form factor) is selected in the range from 3.5 to 2500, preferably from 5 to 500, and more preferably from 5 to 150.

The fibres usable in the composition of the invention can be fibres of synthetic or natural, mineral or organic origin. They can be short or long, individual or organized for example plaited, hollow or solid. They can be of any shape and notably of circular or polygonal (square, hexagonal or octagonal) section depending on the specific application envisaged. In particular, their ends are blunted and/or polished to avoid injury.

In particular, the fibres have a length in the range from 1 μm to 10 mm, preferably from 0.1 mm to 5 mm and more preferably from 0.3 mm to 3 mm. Their section can be contained in a circle with a diameter in the range from 2 nm to 500 μm, preferably in the range from 100 nm to 100 μm and more preferably from 1 μm to 50 μm. The weight or fineness of fibres is often given in denier or decitex and represents the weight in grains for 9 km of thread. Preferably, the fibres according to the invention have a fineness selected in the range from 0.01 to 10 denier, preferably from 0.1 to 2 denier and more preferably from 0.3 to 0.7 denier.

The fibres usable in the compositions according to the invention can be selected from rigid or non-rigid fibres, and they can be of synthetic or natural, mineral or organic origin.

Moreover, the fibres can be surface-treated or not, coated or not, coloured or not coloured.

As fibres for use in the compositions according to the invention, we may mention the non-rigid fibres such as fibres of polyamide (Nylon®) or rigid fibres such as polyimide-amide fibres such as those sold under the designations KERMEL®, KERMEL TECH® by the company RHODIA or poly-(p-phenylene-terephthalamide) (or aramid) fibres notably sold under the designation Kevlar® by the company DUPONT DE NEMOURS.

The fibres can be present at a content in the range from 0.0.1 to 10 wt. %, relative to the total weight of the composition, in particular from 0.1 to 5 wt. %, and more particularly from 0.3 to 3 wt. %.

The compositions according to the invention can further comprise any cosmetic active ingredient such as the active ingredients selected from antioxidants, preservatives, perfumes, bactericides, antiperspirants, neutralizing agents, emollients, hydrating agents, thickeners, trace elements, sequestering agents, alkalizing or acidifying agents, hydrophilic or lipophilic active substances, coalescing agents, plasticizers, vitamins, filters in particular sun filters, and mixtures thereof.

Of course, a person skilled in the art will take care to select any additional compounds, and/or their amount, in such a way that the advantageous properties of the composition according to the invention are not, or substantially not, adversely affected by the addition envisaged.

Packaging

The composition according to the invention can be packaged in a container delimiting at least one compartment, which contains said composition, said container being closed with a closure element.

Closure Element

The closure element can be in the form of a detachable stopper, a lid, a cover, a tearable strip, or a capsule, notably of the type having a body fixed to the container and a cap hinged on the body. It can also be in the form of an element providing selective closure of the container, notably a pump or a valve.

Container

The container can be of any suitable shape. It can notably be in the form of a bottle, a tube, a pot, a case, a box, a sachet or a casing.

The container can be combined with an applicator as detailed below, notably in the form of a brush.

The product can be contained directly in the container, or indirectly. As an example, the product can be arranged on an impregnated carrier, notably in the form of a wipe or a pad, and arranged (one or several) in a box or in a sachet. Such a carrier incorporating the product is described for example in application WO 01/03538.

The closure element can be screwed onto the container. Alternatively, the connection between the closure element, and the container is effected otherwise than by screwing, notably by means of a bayonet mechanism, by a snap-fitting mechanism, or by clamping. “Snap-fitting” means in particular any system involving passing over a collar or bead of material by elastic deformation of a portion, notably of the closure element, then by returning to a position that is not elastically stressed of said portion after passing over the collar or bead.

The container can be made at least partly of thermoplastic material. As examples of thermoplastic materials, we may mention polypropylene or polyethylene.

Alternatively, the container is made of non-thermoplastic material, notably of glass or metal (or alloy).

The container can have rigid walls or deformable walls, notably in the form of a tube or of a scent-bottle tube.

The container can comprise means intended for causing or facilitating distribution of the composition. As an example, the container can have deformable walls so as to cause discharge of the composition in response to an overpressure within the container, said overpressure being caused by elastic (or non-elastic) squeezing of the walls of the container.

The container can be equipped with a wiper arranged in the vicinity of the container opening. Said wiper can be used for wiping the applicator, and optionally the rod to which it is attached. Such a wiper is described for example in patent FR 2 792 618.

Applicator

The applicator can be of various forms. It can notably be in the form of a brush having an arrangement of bristles held in place by a twisted thread. A twisted brush of this kind is notably described in U.S. Pat. No. 4,887,622.

It can also be in the form of a comb having a plurality of application elements, obtained notably by moulding. Such combs are described for example in patent FR 2 796 529.

The applicator can be in the form of an artist's brush, as described for example in patent FR 2 722 380.

The applicator can be in the form of a block of foam or of elastomer. The applicator can be free (sponge) or joined to a rod carried by the closure element, such as described for example in U.S. Pat. No. 5,492,426. The applicator can be integral with the container, as described for example in patent FR 2 761 959.

The composition according to the present invention is particularly advantageous when it is used with an applicator of the brush type having an arrangement of bristles held in place by a twisted thread or of the injection-moulded type, namely having an all-in-one core and teeth. In fact, as described above, applicators of this type, and more particularly such applicators having the property of being flexible, can be suitable for application of the compositions according to the present invention, for a period of time, without observing the drawbacks reported above.

In the case of brushes, flexibility results from a number of variables, which may be associated with the nature of bristles, their cross-section, their diameter, their length and their density, among other things.

Within the scope of the present invention, the application elements and more particularly the “flexible” bristles, are those displaying limited resistance to bending, whereas “hard” application elements are defined as being those that display far greater resistance to bending.

As an illustration, and other things being equal, a short application element is harder than a long application element and a thick application element is harder than a thinner application element. Moreover, hollow application elements are more flexible than solid application elements. Generally, for the application elements, there is a diameter below which they are regarded as flexible and above which they are regarded as hard.

For example, in the case of bristles formed from fibres of nylon or of polyester, relatively flexible bristles have a diameter less than 10 hundredths of a millimetre, whereas relatively rigid bristles have a diameter greater than 10 hundredths of a millimetre and generally less than 30 hundredths of a millimetre.

For example, the bristles or teeth can be made of materials of different flexibility. The hardness of these materials can be compared by the Shore hardness values. The bristles can be natural or synthetic. They can be made by extrusion of a plastic, such as PE, PA, notably PA6, PA6/6, PA6/10 or PA6/12, HYTEL®, PEBAX®, silicone, PU, this list not being exhaustive. These application elements can for example have a hardness between 20 Shore A and 40 Shore D.

It is possible to use bristles of circular cross-section or other than circular. For example, bristles of circular section can be used with a diameter between 50 and 300 hundredths of a millimetre.

According to a particular embodiment, a composition according to the invention can be a composition intended to be applied on the eyelashes, also called “mascara”. It can be a make-up composition, a “base-coat” cosmetic composition, a composition to be applied on a base-coat cosmetic composition, also called “top-coat”. The mascara is more particularly intended for the eyelashes of human beings, but also for false eyelashes.

The compositions according to the invention can be manufactured by the known methods generally used in the field of cosmetics.

The invention is illustrated in more detail in the following examples, which are presented for purposes of illustration and do not limit the invention.

### EXAMPLES

In the examples, percentages by weight are expressed relative to the total weight of the composition.

### Examples 1 and 2

### Mascaras with Two Different Fatty Alcohols

| | Example 1 | Example 2 |
|---|---|---|
| Ingredients | wt. % | wt. % |
| beeswax⁽¹⁾ | 4.4 | 4.4 |
| carnauba wax⁽²⁾ | 3.5 | 3.5 |
| paraffin wax⁽³⁾ | 13.9 | 13.9 |
| cetyl alcohol | 4 | 0 |
| behenyl alcohol | 0 | 4 |
| black iron oxide | 7.14 | 7.14 |
| gum arabic | 0.63 | 0.63 |
| potassium cetyl phosphate⁽⁴⁾ | 7 | 7 |
| hydroxyethyl cellulose⁽⁵⁾ | 0.75 | 0.75 |
| acrylate copolymer by weight of raw material⁽⁶⁾ | 5 | 5 |
| preservatives & active ingredients | 4.36 | 4.36 |
| deionized water | Qs | Qs |
| TOTAL | 100 | 100 |

⁽¹⁾WHITE BEESWAX SP 453P marketed by STRAHL & PITSCH
⁽²⁾CARNAUBA WAX SP 63 marketed by STRAHL & PITSCH
⁽³⁾CERAFINE 56/58 PASTILLES marketed by BAERLOCHER
⁽⁴⁾AMPHISOL K marketed by GIVAUDAN
⁽⁵⁾CELLOSIZE QP 4400 H marketed by AMERCHOL (DOW CHEMICAL)
⁽⁶⁾DAITOSOL 5000 AD marketed by DAITO KASEI KOGYO

After manufacture, these mascaras are submitted to conditions of accelerated ageing at 45° C. Texture analysis is performed according to the protocol described previously.

It can be seen that the formulation comprising behenyl alcohol has a texture value greater than 50 g, which ensures that it has good extending properties. Its texture value does not need to be lowered to compensate for the increase in texture value over time.

Moreover, the relative change in penetration value is lower in the formula comprising behenyl alcohol.

| | Example 1 | Example 2 |
|---|---|---|
| | cetyl alcohol | behenyl alcohol |
| Penetration value | 38 | 75 |
| T0 (g) | | |
| Penetration value | 102 | 78 |
| after 60 d at 45° C. | | |
| (g) | | |
| Relative change | +268% | +4% |

The formula in example 2, relatively less thickening than the formula in example 1, can be used with a softer mascara brush than the formula in example 1. This brush can for example have a diameter of 9 mm with 340 bristles of polyester elastomer of diameter 13/100. After 60 days of accelerated ageing at 45° C., the quality of make-up obtained by application of the formula in example 2 is still satisfactory with the brush described above. In particular, the “Christmas tree” effect or flattening of the bristles after ageing for 2 months at 45° C. is not seen when this brush is immersed in example 2 (behenyl alcohol). In contrast, the “Christmas tree” effect is seen when this same brush is immersed in example 1 (cetyl alcohol) after 2 months at 45° C.

### Examples 3 and 4

### Mascaras with Two Different Fatty Alcohols

| | Example 3 | Example 4 |
|---|---|---|
| Ingredients | wt. % | wt. % |
| beeswax⁽¹⁾ | 4.4 | 4.4 |
| carnauba wax⁽²⁾ | 3.5 | 3.5 |
| paraffin wax⁽³⁾ | 13.9 | 13.9 |
| cetyl alcohol | 2 | 0 |
| behenyl alcohol | 0 | 2 |
| black iron oxide | 7.14 | 7.14 |
| Steareth-2 | 2.1 | 2.1 |
| gum arabic | 0.63 | 0.63 |
| potassium cetyl phosphate⁽⁴⁾ | 7 | 7 |
| hydroxyethyl cellulose⁽⁵⁾ | 0.75 | 0.75 |
| acrylate copolymer⁽⁶⁾ | 5 | 5 |
| preservatives & active ingredients | 4.36 | 4.36 |
| deionized water | Qs | Qs |
| TOTAL | 100 | 100 |

⁽¹⁾WHITE BEESWAX SP 453P marketed by STRAHL & PITSCH
⁽²⁾CARNAUBA WAX SP 63 marketed by STRAHL & PITSCH
⁽³⁾CERAFINE 56/58 PASTILLES marketed by BAERLOCHER
⁽⁴⁾AMPHISOL K marketed by GIVAUDAN
⁽⁵⁾CELLOSIZE QP 4400 H marketed by AMERCHOL (DOW CHEMICAL)
⁽⁶⁾DAITOSOL 5000 AD marketed by DAITO KASEI KOGYO

After manufacture, these mascaras are submitted to conditions of accelerated ageing at 45° C. The texture value is measured according to the protocol described previously.

It can be seen that the relative change in penetration value is lower in the formula comprising behenyl alcohol.

| | Example 3 | Example 4 |
|---|---|---|
| | cetyl alcohol | behenyl alcohol |
| Penetration value | 45 | 117 |
| T0 (g) | | |
| Penetration value | 116 | 116 |
| after 60 d at 45° C. | | |
| (g) | | |
| Relative change | +258% | −1% |

The formulation in example 4 therefore displays better relative stability of texture than the reference formula in example 3 (comparative).

1. Cosmetic composition for make-up and/or care of keratin fibres comprising at least one emulsifying system free from triethanolamine stearate, characterized in that it contains at least one pigment and behenyl alcohol, and in that the texture value measured by texture analysis, according to the method of measurement of texture described in the present application, counting from preparation of said composition, namely 24 hours after manufacture of the composition, is greater than 20 g at room temperature,
said behenyl alcohol being present at a content greater than or equal to 1 wt. % relative to the total weight of the composition,
said emulsifying system comprising at least one surfactant selected from:
i) an alkali metal alkyl phosphate or phosphine oxide of formula (R—O)ₙ—P═O—(O⁻M)ₘ with R representing a linear or branched C₈-C₂₂ alkyl group, such as cetyl, n being equal to 1, 2 or 3 and m being equal to 0, 1 or 2, with m+n being equal to 3 and M representing a hydrogen atom or an alkali metal or alkaline-earth metal, preferably n=1 and m=2, and M is an alkali metal, such as sodium or potassium,
ii) a polyethoxylated alcohol of formula R′—(OCH₂CH₂)ₚ—OH with R′ representing a linear or branched C₁-C₃₀ alkyl and in particular represents CH₃—(CH₂)₁₇⁻ and p representing an integer between 1 and 30 inclusive, preferably between 2 and 20; such as steareth-20 and steareth-2,
iii) a salt of glutamic acid of formula R—CONH—C(COO⁻M)—C₂H₄—COO-M′ with R representing a linear or branched C₈-C₂₂ alkyl group such as stearyl and M′ representing an alkali metal or alkaline-earth metal, and
iv) an alkyl glucoside obtained by condensation of glucose and of linear or branched C₈-C₂₂ fatty alcohols such as a cetyl and stearyl mixture called cetearyl.

2. Composition for make-up and/or care of keratin fibres according to claim 1, said emulsifying system comprising at least one surfactant according to point (i) and/or at least one surfactant according to point (iii), as well as optionally at least one surfactant according to point (ii) and/or at least one surfactant according to point (iv) and/or at least one fatty alcohol comprising from 10 to 26 carbon atoms, preferably from 10 to 24 carbon atoms, and more preferably from 12 to 21 carbon atoms

3. Cosmetic composition for make-up and/or care of keratin fibres according to claim 1, wherein the texture value measured by texture analysis counting from preparation of said composition, namely 24 hours after manufacture of the composition, is greater than 30 g at room temperature, or even greater than 60 g, or greater than 70 g.

4. Cosmetic composition for make-up and/or care of keratin fibres according to claim 1, wherein the texture value measured by texture analysis after a period of 60 days at 45° C. is less than or equal to 100 g, or even less than or equal to 90 g.

5. Cosmetic composition for make-up and/or care of keratin fibres according to claim 1, wherein the change in texture measured by texture analysis in a period of 60 days at 45° C. counting from preparation of said composition, namely 24 hours after manufacture of the composition, is less than 100%, the change in texture being defined by:
$$\frac{{T}_{60 j} - {T}_{0}}{{T}_{0}} \times 100$$
where T₆₀ⱼ is the measurement from texture analysis at 60 days, and T₀ is the measurement from texture analysis 24 hours after manufacture of the composition.

6. Cosmetic composition according to claim 1, wherein the change in texture measured by texture analysis in a period of 60 days at 45° C. counting from preparation of said composition is less than 70%, or even less than 60%, or even less than 50%, for example less than 20%, 10%, or 5% and more particularly at a content in the range from 0.3 to 20 wt. %, notably from 0.5 to 10 wt. % and for example from 0.7 to 7%, or even from 1 to 6 wt. % relative to the total weight of the composition.

7. Cosmetic composition according to claim 1, comprising an aqueous continuous phase.

8. Cosmetic composition according to claim 1, wherein behenyl alcohol is present at a content greater than or equal to 2 wt. %, relative to the total weight of the composition.

9. Cosmetic composition according to claim 1, wherein the emulsifying system comprises at least one ethoxylated and/or propoxylated ether which can comprise from 1 to 150 ethoxylated and/or propoxylated groups, of C₈-C₂₄, and preferably C₁₂-C₁₈ alcohol, such as ethoxylated ether of stearyl alcohol with 2 ethoxylated groups, and an alkali metal alkyl phosphate or phosphine oxide of formula (R—O)ₙ—P═O—(O⁻M)ₘ with R representing a linear or branched C₈-C₂₂ alkyl group, such as cetyl, n being equal to 1, 2 or 3 and m being equal to 0, 1 or 2, with m+n being equal to 3 and M representing a hydrogen atom or an alkali metal or alkaline-earth metal, preferably n=1 and m=2, and M is an alkali metal, such as sodium or potassium as surfactants.

10. Cosmetic composition according to claim 1, wherein the emulsifying system comprises at least one surfactant selected from potassium cetyl phosphate, steareth-2, steareth-20 and mixture thereof.

11. Cosmetic composition according to claim 1, wherein the emulsifying system comprises at least one surfactant selected from sodium stearoyl glutamate, cetearyl glucoside and mixture thereof.

12. Cosmetic composition according to claim 1, wherein the emulsifying system comprises a surfactant with HLB greater than 8 together with a surfactant with HLB less than 8.

13. Cosmetic composition according to claim 1, wherein it further comprises at least one lipophilic structure-forming agent such as waxes, pasty fats and mixtures thereof.

14. Kit for packaging and application comprising a container containing a composition according to claim 1 and an applicator configured for applying said composition on a keratinous material, and in particular on keratin fibres, such as the eyelashes or eyebrows, said applicator comprising application elements, such as bristles or teeth, having a hardness between 20 Shore A and 40 Shore D.

15. Method of coating of keratin fibres, such as the eyelashes or eyebrows, comprising a stage of application of a composition according to claim 1 on said keratin fibres.
